(19) United States
(12) Patent Application Publication
(10) Pub. No.: US 2006/0211604 A1
(43) Pub. Date: Sep. 21, 2006

(54) Methods for treating inflammatory disorders using regulators of microvessel dilations

(75) Inventor: Steven J. Mentzer, Boston, MA (US)

Correspondence Address: Helen C. Lockhart;Wolf, Greenfield & Sacks, P.C., 600 Atlantic Avenue, Boston, MA, 02210, US

(73) Assignee: The Brigham and Women's Hospital, Inc., Boston, MA (US)

(21) Appl. No.: 11/288,030

(22) Filed: Nov. 28, 2005

Related U.S. Application Data
(60) Provisional application No. 60/636,814, filed on Dec. 16, 2004.
(60) Provisional application No. 60/631,094, filed on Nov. 26, 2004.

(51) Int. Cl. A61K 38/54; A61K 38/17; C12Q 1/00

(52) U.S. Cl. 514/8; 514/12; 435/4

(57) ABSTRACT

The invention relates to methods for treating inflammatory diseases. Methods for analyzing microcirculation structural changes are also provided.

### RELATED APPLICATIONS

This application claims priority under 35 U.S.C. §119(e) to U.S. Provisional Application Ser. No. 60/636,814, filed Dec. 16, 2004; U.S. Provisional Application Ser. No. 60/631,094, filed Nov. 26, 2004, and entitled “METHODS FOR TREATING INFLAMMATORY DISORDERS USING REGULATORS OF MICROVESSEL DILATIONS”, the contents of which are herein incorporated by reference in their entirety.

### FEDERALLY SPONSORED RESEARCH

This invention was made with at least in part using Government support under NIT grant HL47078. Accordingly, the Government may have certain rights in this invention.

### FIELD OF THE INVENTION

The present invention relates generally to methods of treating inflammatory disorders such as autoimmune disease, as well as methods for analyzing micro circulation.

### BACKGROUND OF THE INVENTION

When recirculating lymphocytes migrate from the microcirculation to the extravascular site of inflammation, they must overcome the mechanical forces produced by blood flow. Blood flowing across the vascular endothelium creates shear forces at the endothelial boundary that are dependent on both flow velocity and vessel geometry. These shear forces disrupt the lymphocyte-endothelial cell adhesions necessary for transmigration. For more than a decade, the prevailing hypothesis has been that lymphocyte transmigration out of the inflammatory microcirculation occurs because hemodynamic stresses in the microcirculation are overcome by a multi-step sequence of adhesive interactions between lymphocytes and endothelial cells. This hypothesis, however, leaves an unresolved discrepancy between microvascular shear stress (on the order of 10-100 dyn/cm²) and lymphocyte adhesivity (minimal adhesion>1 dyn/cm²).

### SUMMARY OF THE INVENTION

The invention is based on several discoveries relating to anatomical changes within the microcirculation that occur and result in the promotion of lymphocyte transmigration across the vascular endothelium.

Herein, it is shown that lymphocyte slowing and transmigration in the skin, bowel, colon, and lung are associated with acute dilated vascular segments termed “acute microvessel dilatations.” These acute microvessel dilations may or may not include focal microvessel dilations (“microangiectasias”). Acute microvessel dilatations are shown to be associated with a proliferative and/or remodeled endothelium. It has been discovered that downstream vessels are frequently dilated as well. Additionally it has been discovered that at least in some tissues these microvascular changes are associated with neoangiogenesis, involving endothelial cell proliferation. These changes are particularly evident in the colon. The vascular dilatations observed in the lung appear to involve the bronchial arteries more than the pulmonary circulation.

The dependence of acute microvessel dilatation formation on structural adaptations of the vascular endothelium has led to the discovery of new therapeutic interventions for the treatment of inflammatory disorders in these tissues. Thus, in the present invention, methods for both the treatment and prevention of pathologies involving lymphocytic inflammation are disclosed.

Inhibitors of lymphocyte cell-cell adhesion molecules (for example, LFA-1, ICAM-1, and L-Selectin) that have shown poor success in the past for inhibiting lymphocyte transmigration can be combined with inhibitors of acute microvessel dilatation formation, such as anti-angiogenic compounds that inhibit endothelial growth. Disclosed herein are methods for treatment of lymphocytic inflammation using inhibitors of angiogenesis alone or in combination with anti-adhesion compounds.

It has also been discovered that the time course and intensity of the inflammatory response correlate with the development of these vascular changes. The acute changes occur within 4 days. Repeated inflammatory challenges appear to be involved in chronic changes associated with autoimmune diseases in the skin, gut and lung.

In some aspects of the invention a method for treating a subject having a disease involving inflammation by administering an inhibitor of dilatation or an inhibitor of angiogenesis in an amount sufficient to inhibit the formation of acute microvessel dilations is provided. In some embodiments the subject has an autoimmune disease, such as an autoimmune disease of the lung, e.g., idiopathic pulmonary fibrosis and interstitial lung disease, Crohn's disease or ulcerative colitis. In other embodiments the subject has or is at risk of transplant rejection.

Optionally the method may also involve administering an inhibitor of lymphocyte cell-cell adhesion. Inhibitors of lymphocyte cell-cell adhesion include but are not limited to inhibitors of one of LFA-1, CAM 1, and L-selectin.

In one embodiment the inhibitor of dilatation is an inhibitor of angiogenesis. In other embodiments the inhibitor of dilatation includes, for instance, inhibitors of BMPs, such as inhibitors of TGFβ, cell cycle inhibitors, inhibitors of endoglin receptor and inhibitors of angiogenesis.

According to another aspect of the invention a method for treating a subject having an inflammatory bowel disease by administering an inhibitor of angiogenesis in an amount sufficient to treat the inflammatory bowel disease is provided. Optionally the method may also involve administering an inhibitor of lymphocyte cell-cell adhesion. Inhibitors of lymphocyte cell-cell adhesion include but are not limited to inhibitors of one of LFA-1, CAM 1, and L-selectin. In some embodiments the inflammatory bowel disease is Crohn's disease or ulcerative colitis.

A method for analyzing microcirculation structural changes by labeling systemic microcirculation with a lipophilic carbocyanine tracer and performing fluorescence microscopy to analyze the microcirculation structural changes is provided according to other aspects of the invention. The structural changes may be acute or chronic. In one embodiment the method is combined with a method of scanning electron microscopy.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The figures are illustrative only and are not required for enablement of the invention disclosed herein.

FIG. 1 depicts fluorescence photomicrographs of lipophilic carbocyanine vessel painting of the A) retina, B) skin, C) lung, and D) colon. The retina and skin are 3 mm thick sections and the lung and colon 5 um thin sections. The skin and colon were counterstained with the blue fluorescent dye DAPI (bar=100 μm).

FIG. 2 is a comparison of the architecture of the colonic submucosal architecture using A) fluorescent vessel painting, and B) 2D-SEM. The vessel painted sample (A) was processed a thick section (whole mount) of the colonic wall to produce a projection comparable to that obtained with the corrosion casted SEM image (B). The spatial resolution of the fluorescent vessel painted images was limited in the 10-20 μm range (arrows: bars=100 μm).

FIG. 3 is a comparison of interbranch angles measured on images obtained by 2D-SEM (SEM), fluorescent vessel painting (VP) and 3D-SEM. The measured angles are presented as A) a cumulative frequency histogram and B) a box chart. The box chart (B) shows the 25-75 percentile with 2 standard deviations of the mean delineated by error bars. The mean interbranch angles (small square) were SEM=99, VP=109, and 3D-SEM=90.

FIG. 4 is a comparison of interbranch distance measured on images obtained by 2D-SEM (SEM), fluorescent vessel painting (VP) and 3D-SEM. The measured distances are presented as A) a cumulative frequency histogram and B) a box chart. The box chart (B) shows the 25-75 percentile with 2 standard deviations of the mean delineated by error bars. The mean interbranch distances (small square) were SEM=41, VP=53, and 3D-SEM=36.

FIG. 5 is a comparison of vessel diameter measured on images obtained by 2D-SEM (SEM), fluorescent vessel painting (VP) and 3D-SEM. The measured distances are presented as A) a cumulative frequency histogram and B) a box chart. The box chart (B) shows the 25-75 percentile with 2 standard deviations of the mean delineated by error bars. The mean vessel diameter (small square) were SEM=10.4, VP=12.7, and 3D-SEM=9.1.

FIG. 6 depicts relative signal intensity and light dispersion of 10 um diameter vessels after fluorescent vessel painting. The fluorescence of 7 randomly chosen vessel segments in the colonic submucosal plexus was measured by a 50 μm wide linescan located at the vessel midpoint and oriented orthogonal to the vessel axis. The arrows indicate the predicted vessel width based on 3D-SEM.

FIG. 7A is a Box chart of lymphocyte accumulation into the skin using the optical volume fractionator (OVF) method (N=6). Lymphocytic inflammation was triggered by the intrabronchial instillation of the aqueous form of TNP (100 μl, 5% TNBS) and epicutaneous application (300 μl, 7% TNBS). The skin was quick frozen prior to aldehyde fixation and H&E staining (He et al., 2002). The tissue was stereologically sampled and processed (Box: 25-75% range. Error bars: 5-95% range; p<0.0001 by Student's T-test).

FIG. 7B is a Fluorescence micrograph of lymphocyte infiltration into the skin and lung triggered by the peptide-hapten after intrabronchial instillation of the aqueous form of TNP (100 μl, 5% TNBS) and epicutaneous application (300 μl, 7% TNBS). Two differentially labeled lymphocyte populations were infused at 72 hours; lungs and skin were harvested at 96 hours after the application of antigen. The tissue was counterstained with DAPI and aldehyde fixed for serial comparisons.

FIG. 7C is a topographic density map showing lymphocyte accumulation in the 5% oxazalone-stimulated skin harvested at 96 hours. The “pulse” of 10¹⁰ CMFDA fluorescently-labeled lymphocytes was injected 18 hours prior to harvest. The contour lines represent scale space significance at various bandwidths. Since the superficial vascular plexus is 2D, the contour plot is shown in the plane of the superficial plexus.

FIG. 8 depicts tracking fluorescently labeled lymphocyte migrating through a microangiectasia in the oxazolone-stimulated microcirculation 96 hours after antigen exposure. The migratory path of three fluorescently labeled intravascular lymphocytes. The symbols show the location of the intravascular lymphocyte during each frame (33 msec intervals) of the video sequence

FIG. 9 is a scanning electron microscopy image of the cecum with MAE (microangiectasias).

FIG. 10 is a scanning electron microscopy image of the descending colon with vascular dilations.

FIG. 11 is a scanning electron microscopy image of the ascending colon with MAE.

FIG. 12 is a scanning electron microscopy image of control pulmonary vessels.

FIG. 13 is a scanning electron microscopy image of test dilated pulmonary vessels.

FIG. 14 illustrates residence times of lymphocytes in a 400 μm×400 μm area of oxazolone-stimulated skin 96 hours after the application of epicutaneous antigen. Lymphocytes obtained from the draining efferent lymph were fluorescently labeled and injected into the carotid artery at the origin of the external auricular artery. As the labeled lymphocytes passed through the inflamed ear, their movements were tracked using an epifluorescence videomicroscopy system. The data represents a 30 second recording of a single injection of fluorescently labeled lymphocytes. The location and length of time of lymphocytes with a negligible axial velocity is shown. To facilitate presentation, 4 lymphocytes with residence times greater than 2.5 sec are not shown (longest residence time 12 seconds). All lymphocytes returned to the flow stream.

FIG. 15(A) is a scanning electron micrograph of a microangiectasia in the oxazolone stimulated skin 96 hours after the application of antigen (bar=25 μm). FIG. 15(B) illustrates a computational flow model of the microangiectasia demonstrating both simple (filled arrow heads) and complex (open arrow heads) trajectories. Flow condition: Re˜10⁻². Note that a near-zero Re(˜10⁻⁸) flow was also calculated as a control case to ensure that an inertialess flow would be smooth and uniaxial in the microangiectasia (data not shown).

FIG. 16 is a graph depicting residence time profiles of individual lymphocytes passing through the modeled microangiectasia. The axial location of migrating cells (Y axis) is plotted as a function of the cumulative time within the microangiectasia (X axis). Cells with a simple trajectory rapidly passed through the microangiectasia with short residence times (labeled “S”); cells with a complex or looping trajectory demonstrated prolonged residence times (red lines). A cell trapped by rotation flow near the exit of the microangiectasias demonstrated retrograde or looping motion (labeled “L”).

FIG. 17 is a graph illustrating flow paths of individual lymphocytes moving through a single microangiectasia. The intravital epi-fluorescence videomicroscopy system recorded cell movements after the intra-arterial injection of fluorescently labeled lymphocytes. After time-base correction and routine distance calibration, the locations of individual cells were plotted at 33 msec intervals. Most cells demonstrated a smooth flow path through the microangiectasia (small filled circles). These cells slowed as they passed through the microangiectasia but demonstrated a uniaxial flow path. Other cells demonstrated increased residence time (clustered large circles; open arrow head) and even retrograde movements (small arrows) prior to returning to the flow stream. Occasional cells moved radially to the vessel wall and appeared to transmigrate (open arrow).

FIG. 18 shows two graphs, illustrating the time course of TNBS-induced colitis reflected by (A) changes in total body weight and (B) the infiltration of perivascular mononuclear cells. The weight of the mice was expressed as a percentage of their baseline body weight (grams). The number of infiltrating mononuclear cells per 250 μm×250 μm grid was measured by image analysis of serial histologic sections vertically sampled through the wall of the colon. Error bars reflect one standard deviation.

FIG. 19 shows two scanning electron microscopy images of the normal architecture of the colonic microcirculation in the mouse. (A) The polygonal mucosal plexus (labeled “MP”) is supplied by ascending arterioles (labeled “AA”) and parallel descending viens. (B) The relatively uniform polygonal mucosal plexus surrounds colonic crypts (bar=200 μm).

FIG. 20 is two distribution graphs showing topography of the TNBS-induced mononuclear infiltrate 96 hours after the instillation of antigen. Serial optical sections of a 1 mm² grid were analyzed in whole mounts of the colon wall for the presence of infiltrating mononuclear cells. The results of representative A) control and B) TNBS-treated mice are shown. The mucosal capillary plexus was arbitrarily defined as 0 (line) with positive numbers extending to the lumenal surface and negative numbers extending to the serosal surface.

FIG. 21 shows two scanning electron microscopy images of corrosion casting and SEM of the colonic mucosal plexus. The microcirculation was casted 96 hours after the transrectal instillation of (A) vehicle control, or (B) TNBS antigen (bar=50 μm).

FIG. 22 is six graphs showing morphometry of microvessels in the colon mucosal plexus was compared in mice treated with TNBS or vehicle control 96 hours after the instillation of antigen. Morphometric measurements, including branch angles (A,B), interbranch distance (C,D), and vessel diameter (E,F), were obtained on images from 3D-SEM and plotted as cumulative frequency histograms (A,C,E) and box charts (B,D,F). The box charts show the 25-75 percentile with 2 standard deviations of the mean delineated by error bars.

FIG. 23 shows cell movements in the mucosal plexus. (A) Time-location map of a mononuclear cell traversing the mucosal plexus. The location of the cell is mapped at 33 msec. Marked variation in cell velocity is noted during mucosal transit (arrow) (bar=80 μm). (B) Comparison of cell velocity measurements in a randomly selected sample of mononuclear cells. The box chart shows the 25-75 percentile with 2 standard deviations of the mean delineated by error bars.

### DETAILED DESCRIPTION

Structural adaptations of the vascular endothelium in acute microvessel dilatation formation is described herein. The discoveries described herein have led to the discovery of new therapeutic interventions for the treatment of inflammatory disorders in tissues, such as skin, gut and lung. Thus, in the present invention, methods for both the treatment and prevention of pathologies involving lymphocytic inflammation are disclosed.

“Acute microvessel dilatations” refers to the occurrence of an abrupt transition from normal to dilated of a local region of vascular tissue that has been found to occur within the microcirculation of mammals. The formation of an “acute microvessel dilatation” is characterized by an acute dilation and in some instances a focal dilation, or ballooning, of the microvessels which may also be associated with dilation of downstream vessels.

A “focal microvessel dilation” (microangiectasias) is an area of focal venular dilation that is found within inflammatory microvasculature. The presence or formation of a “focal microvessel dilatation” is defined by one or more of i) a localized increase of at least two-fold in microvessel diameter across any cross-sectional orientation of the vessel—on either side of a “focal microvessel dilatation” the diameter of the microvessel is in the normal range of 10-20 μm, ii) the presence of perivascular or extravascular lymphocytes or lymphocyte transmigration, iii) a localized decrease in blood cell flow velocity (which is necessarily accompanied by a decrease in wall shear stress), and iv) a proliferative/hypertrophic vascular endothelium. A “focal microvessel dilatation” can range up to about 90 μm in diameter. Histological studies show that “focal microvessel dilatations” are associated with a proliferative endothelium. The “focal microvessel dilatations” tend to be located at about 100 μm intervals apart from each other in regions of inflammation, for example, in skin tissue. Morphological studies demonstrate that the area of vessel dilation has frequently greater than a 2-fold increase in lumenal diameter, for example, 3-fold or more. The increase in the lumenal diameter of focal microvessel dilatations locally reduces the wall shear stress to below 3 dyn/cm². Normal, non dilated microvessels have a wall shear stress on the order of 15-20 dyn/cm².

Without being bound to any one mechanism, it is believed that the localized reduction in blood cell flow velocity, complex flow patterns, and the resulting localized reduction in wall shear stress of acute microvessel dilatation facilitate lymphocyte transmigration across the endothelium to an extravascular site of inflammation.

As used herein, “formation of a focal microvessel dilatation” (or “formation of a microangiectasia”) can be defined by the presence of at least one of the definitional characteristics of a focal microvessel dilatation. The “formation of a focal microvessel dilatation” is “detected” by the observation of at least one of the definitional characteristics.

As used herein, the term “acute” means that the diameter of the vessel changes abruptly, rather than gradually. An abrupt change is a change wherein the diameter of the vessel at least doubles over a length of the vessel no greater than the original or minimal diameter of the vessel before the change in diameter.

As used herein, the term “inflammation” refers to the presence of tissue damage in an individual. For example, the tissue damage can result from autoimmune processes, microbial infection, tissue or organ allograft rejection, neoplasia, idiopathic diseases or such injurious external influences as heat, cold, radiant energy, electrical or chemical stimuli, or mechanical trauma. Regardless of the cause, the inflammatory response generally comprises an intricate set of functional and cellular changes, involving modifications to microcirculation (including acute and/or focal microvessel dilatation formation), accumulation of fluids, and the influx and activation of inflammatory cells (e.g. lymphocytes).

As described herein, an “increase in diameter” of a microvessel represents at least a 2 fold increase in diameter in any cross sectional dimension as compared to the normal microvessel diameter range of 10-20 μm.

As described herein, a “reduction in blood cell flow velocity” refers to at least a 10-fold reduction in velocity as compared to that observed in undilated microvessels As described herein, a “decrease in wall shear stress” is indicative of focal microvessel dilatation formation. Herein, a “decrease” is considered greater than a 5-fold decrease in wall shear stress as compared to the wall shear stress of normal microvessels, which ranges from 20 to 100 dyn/cm².

As described herein, “extravascular lymphocyte accumulation” refers to the presence of regional lymphocytic perivascular clusters of lymphocytes, which is indicative of the presence of a focal microvessel dilatation. The presence of lymphocytic perivascular clusters may be measured by injecting labeled lymphocytes into the microcirculation at discrete time points. A “difference” in the accumulation of extravascular lymphocytes is an increase or decrease in extravascular lymphocyte accumulation.

An “increase in extravascular lymphocyte accumulation” means at least a 2 fold increase, preferably at least a 3-, 5-, 10-fold or greater increase in the number of extravascular lymphocytes detected in a tissue region exposed to a test compound relative to a region not exposed to that compound.

A “decrease in extravascular lymphocyte accumulation” means at least a 2-fold decrease, and preferably at least a 3-, 5-, 10-fold or greater decrease in the number of extravascular lymphocytes in a tissue region contacted with a test compound and an inducer of inflammation, relative to a tissue region contacted with the inducer of inflammation alone.

As used herein, an “increase in lymphocyte transmigration” refers to at least a 10-fold increase in the transmigration frequency of lymphocytes across the endothelium in comparison to basal level rates which can range from 10²-10³ lymphocytes per minute. As used herein, “endothelial cell proliferation” can refer to endothelial cell division or to a change in size of the endothelial cell. Endothelial cell proliferation can be monitored using cell cycle-specific markers.

As used herein, “reducing the amount of lymphocytic infiltration” refers to preventing lymphocytic transmigration across the microvasculature endothelium such that the rate of transendothelial migration is less than the rate observed in acute rejection which is on the order of more than 10⁶ lymphocytes per minute. Further, “reducing” the amount of lymphocytic infiltration refers to preventing lymphocytic transmigration across the microvasculature endothelium such that lymphocytic inflammation is subdued.

As used herein, “microcirculation” refers to the vascular network lying between the arterioles and venules. The “microcirculation” includes capillaries, metarterioles and arteriovenous anastomoses, venules, and the flow of blood through this network. The “inflammatory microcirculation” refers to areas of the microcirculation where lymphocytes can transmigrate. As used herein, “microvasculature” or “microvessels” refer to venules, capillaries, metarterioles and arteriovenous anastomoses.

As used herein, the modifier “substantially no” reduction or decrease, when applied to an increase, means that there is less than a 5% change in the value being measured relative to a reference, e.g., less than a 5% change in the value being measured in a tissue treated with a compound, relative to that value detected in a tissue not treated with the compound.

A “subject” shall mean a human or vertebrate mammal including but not limited to a dog, cat, horse, cow, pig, sheep, goat, or primate, e.g., monkey.

“Lymphocytic inflammation” can occur in autoimmune disease, graft vs host disease and in viral diseases, such as Herpes Simplex Virus, Varicella, and Herpes Zoster.

Thus, the active agents described herein (i.e., inhibitors of angiogenesis, inhibitors of dilatation, and/or inhibitors of lymphocyte cell-cell adhesion) are useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to autoimmune diseases of the lung, such as idiopathic pulmonary fibrosis and interstitial lung disease, rheumatoid arthritis, Crohn's disease, ulcerative colitis, multiple sclerosis, systemic lupus erythematosus (SLE), transplant rejection, autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

Inflammatory bowel disease is a medical term is used for both Crohn's disease and ulcerative colitis, two diseases in which the immune system attacks the gut (intestine). Inflammatory bowel disease (IBD) is the general name for diseases that cause inflammation in the small intestine and colon. Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis. The inflammation makes the colon empty frequently, causing diarrhea. Ulcers form in places where the inflammation has killed the cells lining the colon; the ulcers bleed and produce pus. Ulcerative colitis can be difficult to diagnose because its symptoms are similar to other intestinal disorders and to another type of IBD called Crohn's disease.

Crohn's disease differs from ulcerative colitis because it causes inflammation deeper within the intestinal wall. Also, Crohn's disease usually occurs in the small intestine, although it can also occur in the mouth, esophagus, stomach, duodenum, large intestine, appendix, and anus.

The active agents are also useful for treating organ transplant rejection. As used herein, “organ transplant rejection” is defined with reference to lymphocyte mediated immune response. In “organ transplant rejection” there is an increase in blood flow to a transplanted organ. The increase in blood flow is associated with increased tissue edema. As used herein, “immunosuppression” refers to prevention of a lymphocyte mediated immune response. As used herein, lymphocytes refer to B or T-cells, wherein, T-cells may be helper T-cells or cytotoxic T-cells.

An inhibitor of dilatation is a compound that prevents any increase in dilatation or slows the process of dilatation of a vessel. These inhibitors include, for instance, inhibitors of BMPs, such as inhibitors of TGFβ, cell cycle inhibitors, inhibitors of endoglin receptor and inhibitors of angiogenesis.

An inhibitor of angiogenesis is any compound that inhibits the promotion or growth of blood vessels or portions thereof. Representative examples of inhibitors of angiogenesis include, but are not limited to, thaloidomide, angiostatin (plasminogen fragment, GenBank Accession No. P20918 (amino acid sequence), antiangiogenic antithrombin III (GenBank Accession No. AH004913 cartilage-derived inhibitor (CDI; Moses & Langer, 1991, J. Cell. Biochem. 47: 230-5), CD59 complement fragment (GenBank Accession No. BT007104), endostatin (collagen XVIII fragment; GenBank Accession No. NMI 30445), fibronectin fragment (GenBank Accession No. BT006856), gro-beta (GenBank Accession No. M36820), heparinases (GenBank Accession No. NMOO6665), heparin hexasaccharide fragment, human chorionic gonadotropin (hCG; GenBank Accession No. V00518), interferon alpha (GenBank Accession No. NMO24013)/beta (GenBank Accession No. NMOO2176)/gamma (GenBank Accession No. AY255837), interferon inducible protein (IP-10), interleukin-12 (GenBank Accession No. NMOO0882), kringle 5 (plasminogen fragment; GenBank Accession No. NMOO0301), metalloproteinase inhibitors (TIMPs; e.g., GenBank Accession Nos. NMOO0362, NMOO3254, NMOO3255), 2-Methoxyestradiol, placental ribonuclease inhibitor, plasminogen activator inhibitor (GenBank Accession No. NM006216), platelet factor-4 (PF4; (GenBank Accession No. NMOO2619), prolactin IRD fragment (GenBank Accession No. NMOO0948), proliferin-related protein (PRP; GenBank Accession No. NMO53364), retinoids, tetrahydrocortisol-S, thrombospondin-I (TSP-1, GenBank Accession No. NMOO3246), transforming growth factor-beta (TGF-β; GenBank Accession No. BT007245), vasculostatin, vasostatin (calreticulin fragment; GenBank Accession No. AY047586), and the like. A number of these factors are available commercially. Inhibitors of angiogenesis may also be small molecules and obtained from natural sources, including: tree bark, fungi, shark muscle and cartilage, sea coral, green tea, and herbs (licorice, ginseng, cumin, garlic).

Inhibitors of lymphocyte cell-cell adhesion refer to any compounds that interfere with the adhesion of cells to one another. Representative inhibitors of lymphocyte cell-cell adhesion include, but are not limited to, “inhibitors” of ICAM-1, LFA-I, and L-selectin. The “inhibitor” may be, for example, a small molecule, antibody, DNA, RNA, or protein. Herein “inhibitor” means any molecule that can either induce an inhibitor or directly inhibit the normal function of cell-cell-adhesion molecules, for example, ICAM-1, LFA-I, and L-selectin.

Herein, an “inhibitor of lymphocyte cell-cell adhesion” can be any molecule that directly binds an adhesion receptor, that inhibits expression of an adhesion receptor, or that inhibits activation of cell adhesion ligands. Example peptide and small molecule cell-cell adhesion inhibitors include, but are not limited to, cyclic ICAM-1-derived peptides (i.e. cIBR and cLAB.L), peptides derived from functional regions of ICAM-1 (i.e. residues 367-394, A1078) and peptides from the alpha- and beta-subunits of LFA Synthetic peptides and peptide-like substances (i.e. peptidomimetics) that possess the amino acid motifs recognized by B1- and B2-integrins may also be used to block leukocyte adhesion. For example, cyclic peptides containing the LDV sequence are potent inhibitors of VLA-4 mediated adhesion. Examples of inhibitors of cell adhesion molecule expression include, but are not limited to, salicylates, methotrexate, and pentoxifylline. In addition, suitable examples of inhibitors of cell adhesion molecule activation, include, but are not limited to, indomethacin, aceclofena, and diclofenac. Active agents can be combined with other therapeutic agents. The active agent and other therapeutic agent may be administered simultaneously or sequentially. When the other therapeutic agents are administered simultaneously they can be administered in the same or separate formulations, but are administered at the same time. The other therapeutic agents are administered sequentially with one another and with active agent, when the administration of the other therapeutic agents and the active agent is temporally separated. The separation in time between the administration of these compounds may be a matter of minutes or it may be longer.

The term “effective amount” refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular active agent being administered, the size of the subject, or the severity of the disease or condition. One of ordinary skill in the art can empirically determine the effective amount of a particular active agent and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds. For any compound described herein the therapeutically effective amount can be initially determined from animal models. A therapeutically effective dose can also be determined from human data for active agents which have been tested in humans and for compounds which are known to exhibit similar pharmacological activities, such as other related active agents. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the active agent can be administered to a subject by any mode that delivers the active agent to the desired surface.

Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds (i.e., active agents, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the active agent (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used. The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the active agent (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the active agent or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Also contemplated herein is pulmonary delivery of the active agents (or derivatives thereof). The active agent (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc.,

St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of active agent (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified active agent may also be prepared in different formulations depending on the type of chemical modification or the type of device employed.

Formulations suitable for use with a nebulizer, either jet or ultrasonic, will typically comprise active agent (or derivative) dissolved in water at a concentration of about 0.1 to 25 mg of biologically active active agent per mL of solution. The formulation may also include a buffer and a simple sugar (e.g., for active agent stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device will generally comprise a finely divided powder containing the active agent (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing active agent (or derivative) and may also include a bulking agent, such as lactose, sorbitol, sucrose, or mannitol in amounts which facilitate dispersal of the powder from the device, e.g., 50 to 90% by weight of the formulation. The active agent (or derivative) should most advantageously be prepared in particulate form with an average particle size of less than 10 μm (or microns), most preferably 0.5 to 5 μm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249:1527-1533, which is incorporated herein by reference.

The active agents and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a active agent and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the active agent, may be provided in particles. Particles as used herein means nano or microparticles (or in some instances larger) which can consist in whole or in part of the active agent or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the active agent in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, 1993, 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

The formation of a focal microvessel dilatation can be determined by the observation of an acute increase in microvessel diameter. Indications of focal microvessel dilatation formation can be obtained from microscopic illumination from a variety of sources (transillumination or epi-illumination). To identify the detailed structure of the microanglectasia focal regions, a corrosion casting technique has been developed that can perfuse the entire microcirculation. This technique was necessary because of the significant arteriovenous interconnections that develop during inflammation. Scanning electron microscopy of the casts has demonstrated focal areas of venular dilatation. In the control circulation, these microvessels are typically 10-20 μm in diameter. The comparable regions examined 96 hours after antigen-stimulation demonstrate balloon-like dilatation up to 50-90 μm in diameter. Herein, focal microvessel dilatation formation can be monitored by the observation of an increase in a regional diameter of the microvasculature. As described herein, an increase represents, at least a 2 fold increase.

The following is an exemplary method for corrosion casting. After systemic heparinization with 750 u/kg intravenous heparin, external auricular arteries are bilaterally cannulated and perfused with approximately 100 cc of 37° C. saline followed by a 2.5% buffered glutaraldehyde solution (Sigma) at pH 7. The casts can be made by perfusion of ear arteries with 100 cc of Mercox (SPI, West Chester Pa.) diluted with 20% methylmethacrylate monomers (Aldrich Chemical, Milwaukee Wis.). After complete polymerization, the ears are harvested and macerated in 5% potassium hydroxide followed by drying and mounting for scamiing electron microscopy. The microvascular corrosion casts can be imaged after coating with gold in Argon atmosphere with a Philips ESEM XL30 scanning electron microscope.

The formation of a focal microvessel dilatation can also be determined by the observation of a decrease in blood cell flow velocity within a focal region of a microvessel. The focal dilation of a microvessel has an impact on the regional microhemodynamics. The effect can be illustrated using a river analogy, a sudden widening of a river, of the relative magnitude of a focal microvessel dilatation, results in a dramatic slowing of any object in the flow stream. Lymphocyte slowing can be monitored by intravital videomicroscopy studies as described in, West et al., 2001, Am. J. Physiol. Heart Circ. 281: 1. To optimize visualization, lymphocytes, red blood cells, neutrophils, or other particles in the size range of these cells are fluorescently labeled. The fluorescent labeling of migratory lymphocytes leaving the antigen stimulated lymph node has allowed the tracking of their migration into the antigen-stimulated skin and lung. Using epi-fluorescence video microscopy, the movement of lymphocytes or other labeled cells or particles in the tissue can be tracked and recorded. These intravital microscopy recordings were the initial demonstration of “recruitment-associated venules.” Using these methods, it has been shown that lymphocytes move through tissues at velocities in excess of 3 μm/msec. In microangiectasia focal regions, the lymphocytes dramatically slow, for example, to velocities less than 0.3 μm/msec.

Herein, a reduction in lymphocyte velocity is at least 10-fold as compared to that normally observed in the absence of a focal microvessel dilatation, which is 3 μm/msec or higher.

Another measure of focal microvessel dilatation formation is the observation of a decrease in wall shear stress of a microvessel. The local dilation of a microvessel has an impact on the wall shear stress. The abrupt decrease in flow velocity in dilated vascular segments produce a marked decrease in shear rates. Wall shear stresses are dependent upon cell velocity and vessel geometry. Flow patterns within the focal microvessel dilatation can be visualized using fluorescent tracers of plasma flow, red cells, lymphocytes and neutrophils. The following parameters are typically monitored when evaluating routine microcirculatory measurements: Diameter (μm), Q (nl/sec), V_{RBC} (μm/sec), V_{lymphocyte} (μm/sec), T_{W} (dyn/cm²), V_{rolling} (μm/sec), Vₘₑₐₙ (μm/sec), and L_{flux} (cell/sec), where Q is the volumetric flow rate, V_{RBC} (μm/sec) is velocity of RBC, V_{lymphocyte} (μm/sec) is velocity of lymphocyte, Tw (dyn/cm²) is the shear stress, V_{rolling} (μm/sec) is a measure of marginated leukocytes, Vₘₑₐₙ (μm/sec) is mean velocity, and L_{flux} (cell/see) is a measure of lymphocyte transmigration. The microhemodynamic assessments in focal microvessel dilatations described herein are based on similar parameters, but the complex flow conditions require computer and mathematical simulations.

Flow patterns and wall shear stress can be assessed in vivo using flow tracers. The analysis of spatial variations in blood flow using fluorescent plasma tracer has several methodological advantages in investigating focal microvessel dilatations. First, the single injection technique has been used in vivo (Burbank et al., 1984, Journal of the American College of Cardiology 4: 308-315) and has been validated in a single input system (Nobis et al., 1985, Microvase. Res. 29: 295). Second, the injection technique permits an assessment of local plasma flow in the focal microvessel dilatations. The direct visualization of the focal microvessel dilatations permits the mapping of flow redistribution at the site of lymphocyte transmigration (West et al., Spatial variation in plasma flow after oxazolone stimulation, Inflammation Res., in press). Third, the direct measurement of emitted light obviated the need for blood sampling and eliminated the errors in downstream venous sampling. Fourth, the use of fluorescence intravital videornicroscopy offers the possibility of multi-color fluorescence labeling of lymphocyte and RBC blood elements (He et al., 2001. J. Histochern. Cytochem. 49: 511). Multi-color labeling may permit the near-simultaneous correlation of lymphocyte flux and blood flow calculations.

The measurement of microcirculatory spatial hemodynamics is obtained by intravital microscopy and motion analysis software algorithms. The movement of the fluorescently labeled cells is recorded as they pass through the tissue using intravital microscopy. Further hemodynamic information can be obtained from plasma marker and labeled red blood cell injections. The videomicroscopy recordings can be analyzed for blood flow and cell velocity as well as cell movements (time-location maps). Specific structural regions of a microcirculation are identified by plasma marker injections as well as temporal area maps (Li X. et al., 1996. Am. J. Respir. Cell Mol. Biol. 14: 398-406; Li X et al., 2001; West C A, et al., 2001c. Am. J. Physiol. Heart Circ. 281: H1742-111750).

At the focal region of a focal microvessel dilatation, lymphocytes transmigrate across the endothelium and form perivascular clusters. The presence of regional lymphocytic perivascular clusters is indicative of the presence of a focal microvessel dilatation. In one embodiment lymphocytes are fluorescently labeled and tracked in vivo for periods much longer than their blood recirculation time of 3 to 5 hours. We have adapted recently developed thiol-reactive cytoplasmic dyes for use in our studies (West C A et al., 2001. J. Histochem-Cytochem. 49: 511). These multi-colored dyes exist in the cytoplasm as fluorescent-peptide adducts so that they are retained in the cytoplasm for more than 72 hours at physiologic temperatures. Furthermore, these dyes are easily distinguishable by fluorescence microscopy, provide effective signal isolation for histologic analysis and are aldehyde fixable.

Second, studies using these cell tracers have demonstrated two significant features of lymphocyte recruitment. First, lymphocyte migration to the peripheral site of antigen stimulation is independent of the lymph node of origin; that is, the frequency of lymphocytes migrating into the antigen-stimulated tissue is very similar whether the lymphocytes are from the stimulated lymph node or the contralateral control lymph node (West C A, et al., 2001. J Immunol 166: 1517-1523). Studies in both the skin and lung demonstrated that lymphocyte recruitment into the tissue occurs in discrete clusters of cells. An explanation for this unexpected observation is that the injection of labeled lymphocytes functions as a “pulse” that enables us to visualize the migration pathway of lymphocytes in inflammation. In most conventional H&E histologic analyses, lymphocytes that have recently transmigrated are indistinguishable from those temporally removed from transmigration. It is speculated that lymphocytes migrating out of the tissue from these discrete areas subsequently percolate through the tissues and leave in the afferent lymph. Consistent with these observations, the longer the delay between injection of the lymphocytes and tissue harvest, the greater the distance from the microcirculation lymphocytes can be observed. These findings are consistent with focal areas of lymphocyte recruitment. Herein, lymphocyte clustering is consistent with focal areas of lymphocyte recruitment, and focal microvessel dilatation formation.

Monitoring endothelial cell proliferation can also be used to assess the formation of focal microvessel dilatations. Endothelial cell proliferation can be monitored by any means known in the art. In one embodiment, endothelial cell proliferation (and inhibition) has been assessed using serial immunohistochemistry of the inflammatory and control microcirculations using standard sereologic sampling techniques.

Immunohistochemistry with the Ki-67 monoclonal antibody was used to detect cell cycle progression. Counterstaining with CD31 or ICAM-2 monoclonal antibodies are used for endothelial localization controls. Intravital microscopy and microvascular corrosion casting with 3-dimensional scanning electron microscopy provide a quantitative measure of the change in venular surface area.

Induction of Focal Microvessel Dilatations Conditions that “permit formation of a focal microvessel dilatation” are the natural physiological conditions present in a mammal. Focal microvessel dilatations can be induced in tissue using peptide-hapten antigens such as, but not limited to, oxazolone and TNP. Both alloantigens (and xenoantigens) and peptide-hapten antigens (e.g. oxazolone and TNBS/TNP) (West C A, et al., 2001. J Immunol 166: 1517-1523) have successfully been used. The evidence to date suggests that the implications for focal microvessel dilatation development are the same for each of these antigens. More details are provided in the Examples below. The present invention is further illustrated by the following Examples, which in no way should be construed as further limiting.

### EXAMPLES

### Example 1

### Vessel Painting of the Microcirculation Using Fluorescent Lipophilic Tracers

The following example describes a new flexible approach to examining acute structural adaptations in the microcirculation. The requirements for tracers used in such methods of quantitative morphometry are stringent: the ideal tracer would be water-soluble with sufficiently low viscosity to label the smallest microvessels. Because detailed morphometric measurements require time-consuming analysis, the tracers should also be retained throughout fixation procedures and persist for prolonged processing. As a result, most morphometric analyses of the microcirculation have relied upon corrosion casting and scanning electron microscopy. Corrosion casting, however, is expensive and technically demanding. These limitations have made corrosion casting impractical for many biological applications.

Attempts to define the morphology of the microcirculation without corrosion casting have focused on lipophilic fluorescent probes. For instance, the inventors have previously used fluorescently labeled liposomes to perfuse microvessels and label endothelial cell membranes in vivo. The advantage of lipophilic dyes is that vascular lining cells provide a high capacity reservoir for the fluorescent tracer. Further, the lateral diffusion of the dye in the cell membrane facilitates the uniform distribution of the dye despite focal application. Among the limitations of this approach are the relative staining inefficiency of the short chained lipid probes and the impermanence of the fluorochrome after aldehyde fixation. To compensate for these limitations, long-chained lipophilic carbocyanines have been developed for long-term cell labeling. These dyes, however, have required the presence of osmolarity regulating agents or the absence of salt to avoid dye precipitation.

In this report, we used lipophilic carbocyanine tracers to label the systemic microcirculation. In contrast to other approaches, sulfonated lipophilic carbocyanine derivatives have improved solubility in water and stability after fixation. The labeling efficiency and the persistence of the tracer after fixation was evaluated in the retina, skin, lung and colon. The technique was validated using morphometric comparisons with corrosion casting and 2-dimensional and 3-dimensional scanning electron microscopy. These studies support the utility of fluorescent vessel painting as a technique in the morphometric study of the microcirculation.

Materials and Methods:

Mice. Male Balb/c mice (Jackson Laboratory, Bar Harbor, Me.), 25-33 g, were used in all experiments. The care of the animals was consistent with guidelines of the American Association for Accreditation of Laboratory Animal Care (Bethesda, Md.).

Lipophilic carbocyanine tracer. The fluorescent dye 1,1-Dioctadecyl-3,3,3,3-tetramethylindocarbocyanine perchlorate was obtained from Sigma (St. Louis, Mo.). The carbocyanine dye was sterilely dissolved in ethanol (6 mg/ml) and stored as a stock solution at 4° C. Immediately prior to infusion, the stock solution was diluted in phosphate buffered saline (PBS) containing glucose (200 mM).

Intravascular flushing and fixation. After systemic heparinization, intraperitoneal anesthesia and thoracotomy, the murine aorta was cannulated with a 2 mm olive tipped cannula (Acufirm 1428LL; Dreieich, Germany) via a left ventriculotomy and the systemic circulation was flushed free of visible blood with PBS warmed to 40° C. After 1 cc of the PBS infusate, the circulation was vented through a right atriotomy. Following the PBS flush, 5 cc of 2.5% glutaraldehyde warmed to 40° C. was infused through the aortic cannula.

Fluorescent vessel painting. Following intravascular fixation, the systemic circulation was perfused with the lipophilic carbocyanine tracer (10-25 ml) at 25° C. Immediately following tracer infusion, the organs were harvested and the tissues dissected in a PBS bath at 25° C. The prepared specimens were placed between glass slides and fixed in 4% formalin overnight. After a brief rinse with distilled water, the specimens were permanently mounted with Vectashield mounting medium (Vector, Burlingame, Calif.). For fluorescence microscopy, the aqueous mounting media with DAPI (4′,6-diamidino-2-phenylindole; 1.5 μg/ml) (Vectashield mounting medium, Vector Laboratories, Burlingame, Calif.) was used in most experiments.

Corrosion casting. Following intravascular fixation, the systemic circulation was perfused with 10-20 ml of Mercox (SPI, West Chester, Pa.) diluted with 20% methyl methacrylate monomers (Aldrich Chemical, Milwaukee, Wis.). After complete polymerization, the tissues were harvested and macerated in 5% potassium hydroxide followed by drying and mounting for scanning electron microscopy. The microvascular corrosion casts were imaged after coating with gold in argon atmosphere with a Philips ESEM XL30 scanning electron microscope. The microvascular corrosion casts were imaged after coating with gold in Argon atmosphere with a Philips ESEM XL30 scanning electron microscope (Eindhoven, Netherlands). Stereo-pair images were obtained using tilt angles from 6 to 20 degrees. Diameters were interactively measured orthogonal to the vessel axis after storage of calibrated images, using AnalySIS software (version 2.1). The quality of the corrosion casts was controlled by semithin light microscopic sections stained with methylene blue. The corrosion casts demonstrated filling of the whole capillary bed from artery to vein without evidence of extravasation or pressure distension.

Digital fluorescence imaging. The fluorescently labeled microvessels were imaged using a Nikon Eclipse TE2000 inverted epifluorescence microscope using Nikon CFI Plan Fluor ELWD10×, 20×, and 40× objectives. An X-Cite (Exfo; Vanier, Canada) 120 watt metal halide light source and a liquid light guide was used to illuminate the tissue samples. Excitation and emission filters (Chroma, Rockingham, Vt.) in separate LEP motorized filter wheels were controlled by a MAC5000 controller (Lud) and MetaMorph software (Universal Imaging, Brandywine, Pa.). The carbocyanine tracer (1,1-Dioctadecyl-3,3,3,3-tetramethylindocarbocyanine perchlorate) and, in some experiments, DAPI (4′,6-diamidino-2-phenylindole) were imaged with 25 nm band pass filters (Omega). The 12-bit fluorescent images were digitally recorded (Cool Snap ES, Roper Scientific, Tuscon, Ariz.) with 1392×1040 pixel resolution. After processing with standard Metamorph filters on a Dell Xeon workstation running Windows XP Professional (Microsoft, Redmond, Wash.), the images were pseudocolored. When DAPI was used as a counterstain, the image was digitally recombined.

Image analysis. Images were processed with the MetaMorph Imaging System 6.1 software (Universal Imaging, Brandwine, Pa.). The 12-bit grayscale images were thresholded and standard distance calibration was performed. The MetaMorph's and caliper applications were used to measure vessel angle, vessel diameter and interbranch distances. The data was logged into Microsoft Excel 2003 (Redmond Wash.) by dynamic data exchange.

Statistical analysis. The statistical analysis was based on measurements in at least three different mice. The unpaired Student's t test for samples of unequal variances was used to calculate statistical significance. The data was expressed as mean+one standard deviation. The significance level for the sample distribution was defined as P<0.01.

Results:

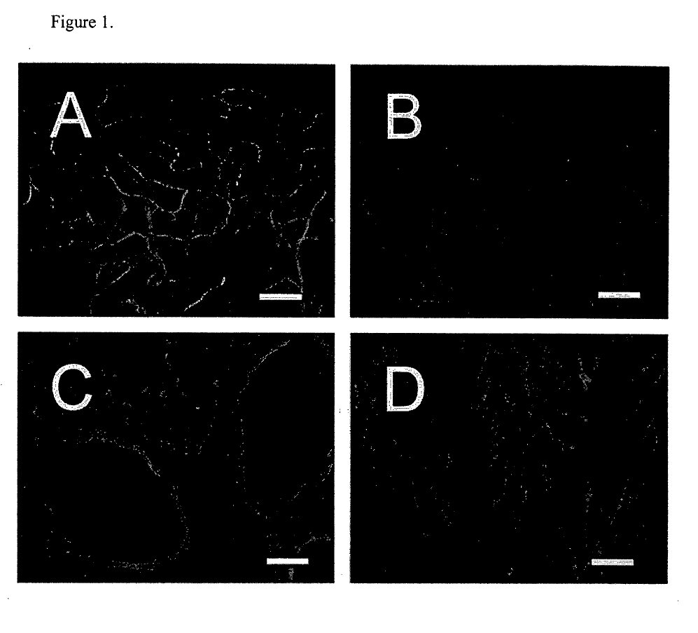

To assess the utility of fluorescent vessel painting, lipophilic carbocyanine dye was used to label the retina, skin, lung, and colon microcirculation (FIG. 1A-D). The long-chained lipophilic carbocyanine tracers uniformly distributed within the microcirculation. In a qualitative comparison with corrosion casting, vessel painting had two advantages. First, vessel painting permitted the use of a tissue counterstain. DAPI, a blue fluorescent dye with a nuclear staining pattern similar to hematoxylin, provided a useful counterstain to delineate tissue architecture (FIG. 1B, D). Second, the lipophilic carbocyanine tracers used in these experiments had significantly lower viscosity than the methylmethacrylate-based casting medium used in corrosion casting. The filling of the high resistance microvessels in the mouse ear was more complete with the low viscosity lipophilic tracer.

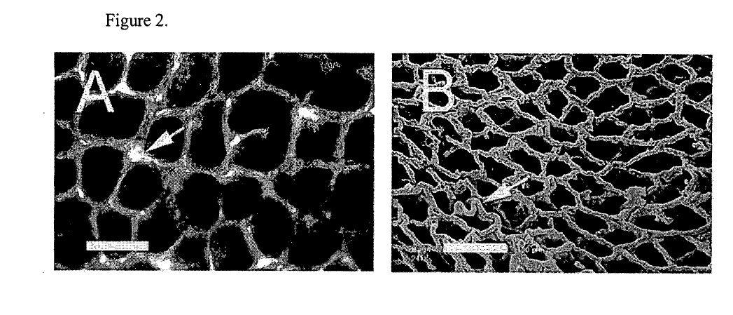

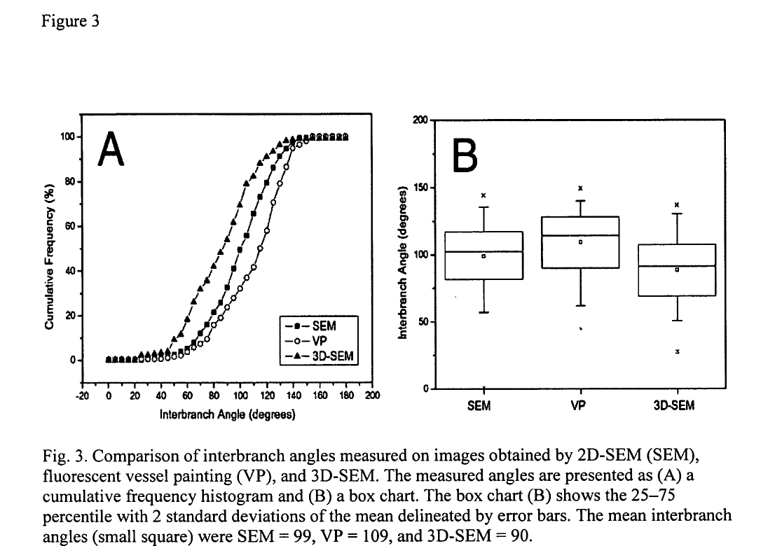

The colonic submucosal plexus is a two-dimensional repetitive architecture that provides an opportunity to compare vessel painting and corrosion casting on multiple topographic features (FIG. 2A, B). The comparison of the interbranch angles in the plexus provided a global measure of tissue distortion that might occur during sample processing. Whereas a qualitative comparison SEM and vessel painting suggested comparable preservation of plexus architecture, a quantitative comparison demonstrated that vessel painting overestimated the interbranch angles measured in the 2D and 3D SEM images (p<0.01) (FIG. 3). A visual analysis suggested that the more limited spatial resolution of fluorescent vessel painting contributed to the inability of vessel painting to discriminate neighboring vessels and interbranch angles (FIG. 2A, B; arrows).

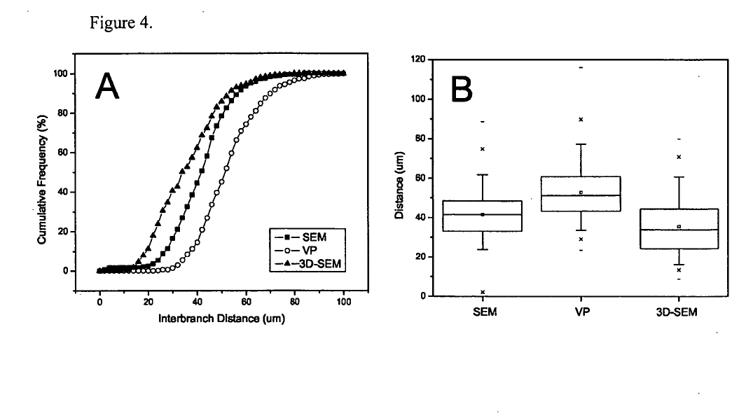

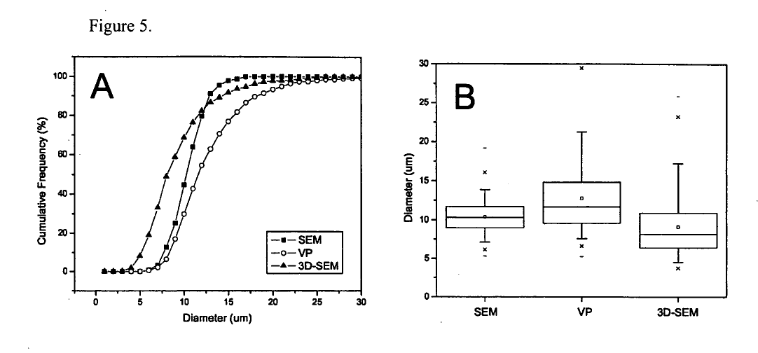

Interbranch distance, a measure of segment length, was similarly assessed by comparing fluorescent vessel painting, 2D and 3D SEM. The fluorescent vessel painting resulted in apparent vessel segment lengths significantly longer than obtained with either 2D or 3D SEM (p<0.01) (FIG. 4). The apparent width of the vessel at the segment midpoint was defined as vessel diameter. Again, fluorescent vessel painting overestimated vessel diameter in comparison with 2D and 3D SEM (p<0.01) (FIG. 5).

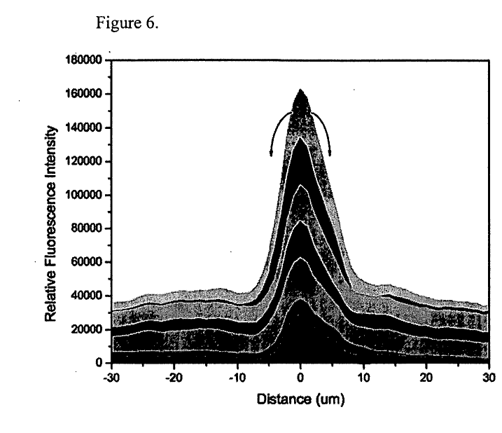

To provide a measure of subjective impact of vessel painting, signal intensity and light dispersion of the fluorescently labeled vessels was measured at the segment midpoint (FIG. 6). Orienting a 100 μm×50 μm linescan orthogonal to the vessel axis, the randomly chosen vessels provide a measure of the vessel fluorescence intensity relative to background fluorescence. Even in this small sample, there appeared to be significant variability in the relative difference between detectable intravascular and extravascular fluorescence. Consistent with the previous measurements (FIG. 5), the dispersion of the fluorescent signal was minimal beyond the expected 10 μm diameter.

In this report, fluorescent vessel painting provided a useful representation of the two dimensional topography of the microcirculation. The interbranch angles, interbranch distances and mid-segment vessel diameters showed a systematic overestimation with fluorescent vessel painting when compared to corrosion casting and SEM. The differences, however, were reproducible and likely secondary to limited spatial resolution. The advantage of fluorescent vessel painting was its relative economy, ease-of-use and low viscosity. The low viscosity was advantage in the high resistance microvessels of the mouse ear. The lipophilic tracer was significantly more efficient in labeling the ear than that methyl methacrylate-based casting media used in corrosion casting.

If anatomic resolution in areas of high microvascular density are difficult to assess using the methodology described herein, complementary methods such as corrosion casting and SEM may be used. In some network architectures, the overlapping emissions from neighboring vessels created the appearance of a single large vessel. In two-dimensions, the images assume a cylindrical microvessel geometry.

The fluorescent vessel “paint” used in these experiments have a charged fluorophore that localizes the probe at the membrane surface and a lipophilic aliphatic “tail” that inserts into the membrane and anchors the probe. The vascular lining cells provide a convenient vehicle for loading cells with lipophilic dyes. Endothelial cell membranes not only adsorb a high concentration of the lipophilic dye, but also facilitate the lateral diffusion of the fluorochrome for more even distribution. Ampiphilic probes with a long alkyl tail (greater than 18 carbons) have demonstrated remarkable stability in long-term tracking studies. Further, the ability of these dyes to persist after standard aldehyde fixation procedures implies their potential utility in detailed morphometric studies as well. The lipophilic tracer used in our studies persisted after more than 6 months of routine storage.

In the past, the disadvantage of the lipophilic carbocyanines were their poor solubility in aqueous media—a property that makes loading the endothelial cells difficult in vivo. The development of enhanced water solubility, combined with their low viscosity, has made these probes particularly useful for labeling of relatively high resistance microvessels. Our experiments suggest that the aqueous viscosity of the lipophilic tracers permits the labeling of the high resistance skin microcirculation when corrosion casting was of limited utility. This viscosity difference suggests that vessel painting can be an important complement to corrosion casting in selected microvascular networks.

### Example 2

### Quantifying Lymphocyte Migration into Peptide-Hapten Stimulated Tissues Using Tissue Cytometry

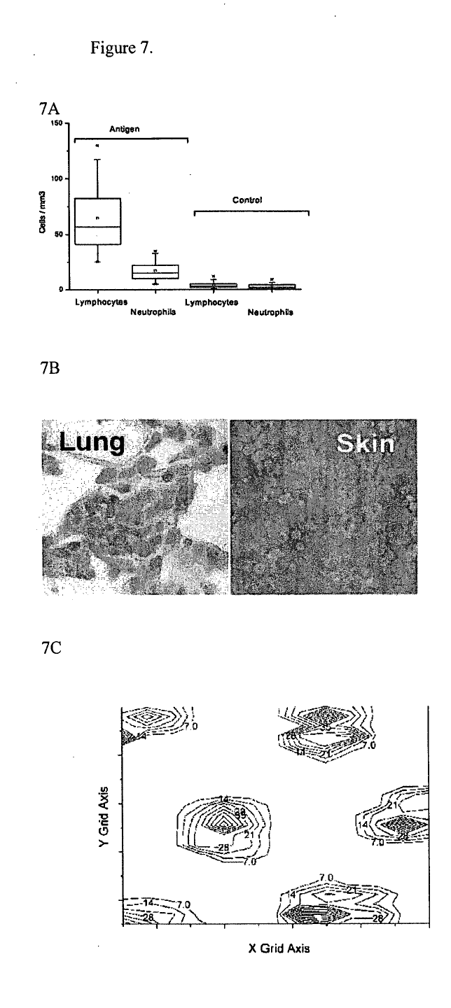

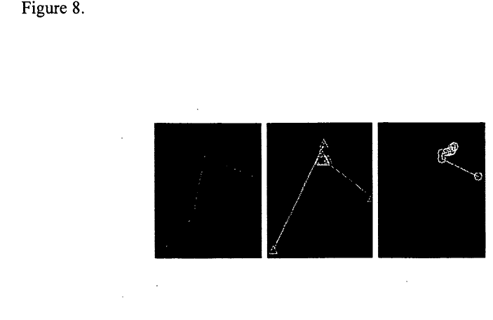

The functional endpoint of microangiectasia development is the accumulation of lymphocytes into the inflammatory tissues. To demonstrate the robust capacity of quantitative morphometry to define the kinetics and topography of lymphocyte accumulation, we studied peptide-hapten stimulated skin 96 hours after the application of antigen. Using design-based sampling techniques, the peptide-hapten stimulated skin demonstrates the selective accumulation of lymphocytes (FIG. 7A).

To demonstrate the focal topography of lymphocyte accumulation in the perivascular tissue, we have developed a “pulse-chase” technique in which fluorescently labeled lymphocytes are injected into the circulation at various intervals prior to harvest (typically 24 hours prior to peak accumulation). The tissue is counterstained with DAPI (hematoxylin-like staining pattern), aldehyde fixed for long-term storage, and examined by fluorescence microscopy. The fluorescence images obtained with different filters (corresponding to visible blue, green and red) are digitally recombined to provide both signal isolation and anatomic resolution (FIG. 7B).

A feature of microangiectasia is the focal recruitment of lymphocytes into the tissue; a consequence of the focal dilatation of the vessel. We apply stereological methods to obtain reproducible and statistically valid density estimates of lymphocyte accumulation in 3D space. To illustrate both the focality of lymphocyte accumulation, as well as the high degree of statistical discrimination, FIG. 7C shows a topographic density map demonstrating focal accumulation of lymphocytes on a 600 μm×600 μm grid overlay. The lines demonstrate scale space significance at various resolutions (bandwidths). The accumulation of lymphocytes at 100 μm intervals corresponds to findings on 3D SEM.

### Example 3

### Polyaxial Flow Paths of Migratory Lymphocytes in Inflammatory Skin

The following example is based at least in part on the discovery that focal dilatations are present at the site of lymphocyte transmigration, correlated with prolonged residence times of lymphocytes, suggesting the possibility of complex plasma flow within the microangiectasias.

To correlate the complex pattern of blood flow associated with structural adaptations of the inflammatory microcirculation, intravascular lymphocyte movement was investigated by use of intravital videomicroscopy and computational flow modeling.

Materials and Methods:

Animals. Randomly bred sheep, weighing 25-35 kg, were used. Sheep were excluded from the analysis if there was any gross or microscopic evidence of dermatitis. The sheep were given free access to food and water. The care of the animals was consistent with guidelines of the American Association for Accreditation of Laboratory Animal Care (Bethesda). As previously described (West, C. A. et al., 2001. J Immunol 166, 1517-23), the sheep ear and neck region was sheared bilaterally and the lanolin was removed with an equal mixture of diethyl ether (Baker, Phillipsburg, N.J.) and ethanol (AAPER, Shelbyville, Ky.). The antigen, a 5% solution of 2-phenyl-4-ethoxymethylene-5-oxazolone (oxazolone; Sigma) was sprayed onto the ear and a localized region of the neck as a 4:1 oxazolone/olive oil mixture by using a syringe and a 23-gauge needle. A vehicle-only control was applied to the contralateral skin.

Intravital microscopy system. The custom-designed epi-illumination system delivered light through the optical system as bright-field, dark-field, or fluorescence illumination. The Nikon epi-achromat objectives were typically used at ×20 magnification (West, C. A et al., 2002. Inflamm Res 51, 572-8). Video of the recorded images was processed through a computer running the Metamorph Imaging System 6.1 (Universal Imaging, Brandywine, Pa.) under Microsoft Windows XP Professional (Redmond, Wash.). Image stacks were routinely created from 12-sec to 5-min video sequences. The image stacks were processed with standard Metamorph filters. After routine distance calibration and thresholding, the “stacked” image sequence was measured by using Metamorph's object tracking and integrated morphometry applications.

Migrating lymphocytes. The prescapular lymph node, with a lymphatic drainage basin including the ear and neck, was used for all efferent lymph duct cannulations as previously described (He, C. et al., 2002. J Appl Physiol 93, 966-973). The lymphocytes were labeled with succinimidyl esters of the mixed isomer preparation of 5-(and 6-)carboxytetramethylrhodamine [5(6)-TAMRA; excitation 540 nm/emission 565 nm; Molecular Probes]. Before labeling, the lymph cells were washed three times in Dulbecco's modified Eagle's medium (DMEM) with 2 g/liter glucose (Sigma) and resuspended in PBS containing 25 μl of the stock 5(6)-TAMRA fluorescent dye. The cells were incubated for 15 min at room temperature and washed in cold DMEM. The cells were resuspended in room-temperature PBS at 0.7-5.0×10⁷ cells per ml before injection into the common carotid arteries proximal to the origin of the external auricular arteries (Su, M. et al., 2003 J Cell Physiol 194, 54-62).

Scanning electron microscopy. After systemic heparinization with 750 units of heparin per kg i.v., the external auricular arteries were bilaterally cannulated and perfused with 100 ml of 37° C. saline followed by a buffered 2.5% glutaraldehyde solution (Sigma) at pH 7.40. After casting of the microcirculation by perfusion of the ear arteries with 100 ml of Mercox (SPI, West Chester, Pa.) diluted with 20% methylmethacrylate monomers (Aldrich) and caustic digestion (Secomb, T. W. et al., 2003. Proc Natl Acad Sci USA 100, 7231-7234), the microvascular corrosion casts were imaged after coating with gold in an argon atmosphere with a Philips ESEM XL30 scanning electron microscope. Stereo-pair images were obtained by using tilt angles from 6° to 20°. The quality of the filling of the corrosion casts was also checked by comparisons with the vascular densities in semithin light microscopic sections stained with methylene blue. The corrosion casts demonstrated filling of the whole capillary bed from artery to vein without evidence of extravasation or pressure distension (Konerding, M. A. et al., 1998. Am J Pathol 152, 1607-16).

Geometric model. Studying 3D scanning electron micrographs (FIG. 2A) (Konerding, M. A. & Steinberg, F., 1989. Prog Clin Biol Res 295, 475-80), the basic configuration of the focally dilated microvessel found in the skin was represented as a balloon-like dilatation located at the hairpin turn in the superficial vascular plexus of the skin (Hughes, T. J. R., 1987. The Finite Element Method-Linear Static and Dynamic Finite Element Analysis. Prentice-Hall, Englewood Cliffs; Huebner, K. H., 1975. The Finite Element Method. John Wiley & Sons, New York). The size of model segment was determined by statistically extracting essential geometric features of microangiectasias from a large pool of detailed morphologic measurements (Secomb, T. W. et al., 2003. Proc Natl Acad Sci USA 100, 7231-7234). Particular attention was paid to the width of microangiectasia (40˜100 μm) relative to the diameters of afferent/efferent vessels (8/9 μm, respectively), and the orientation/acuity of microangiectasias relative to the entry and exit segments. The junction between the afferent/efferent vessels and the microangiectasia were rounded to avoid sharp wall transitions. Three-dimensional (3D) plasma flow domain bounded by the geometric model was discretized into 3D isoparametric finite elements (Hughes, T. J. R., 1987. The Finite Element Method-Linear Static and Dynamic Finite Element Analysis. Prentice-Hall, Englewood Cliffs; Bathe, K. J., 1996. Finite Element Procedures. Prentice-Hall, Englewood Cliffs). The governing flow equations were transformed into the algebraic balance equations for each 8-node finite element using the Galerkin method (Huebner, K. H., 1975. The Finite Element Method. John Wiley & Sons, New York). The number of elements required for the 3D model was about fifty thousand, tested to be sufficient to produce accurate results (data not shown).

Numerical flow calculations. Using this geometric model, blood flow patterns were solved numerically (Donea, J. & Huerta, A., 2002. Finite Element Methods for Flow Problems. John Wiley & Sons, New York; Kojic, M. & Bathe, K. J., in press. Inelastic Analysis of Solids and Structures. Springer-Verlag, New York). Assuming constant viscosity of 2.2 cP (Secomb, T. W. et al., 2003. Proc Natl Acad Sci USA 100, 7231-7234), steady plasma flow field in the geometric model of microangiectasia was defined by the full Navier-Stokes equations and the continuity equation, and was solved numerically in the penalty formulation with stabilized algorithm (Donea, J. & Huerta, A., 2002. Finite Element Methods for Flow Problems. John Wiley & Sons, New York) using a custom-made software package (Kojic, M. & Bathe, K. J., in press. Inelastic Analysis of Solids and Structures. Springer-Verlag, New York). At the inlet of the afferent vessel, constant bulk flow with parabolic velocity profile was imposed; at the outlet of the efferent vessel, a constant-pressure boundary was defined. No slip condition was enforced on all wall surfaces (i.e., endothelial lining cell surface). Hydrodynamic similarity was achieved by matching the Reynolds number Re (given as Re=l\overline{u}/v, where \overline{u} and l are mean bulk velocity and diameter in the afferent vessel, respectively and v is kinematic viscosity of the blood) in the model to Re in the microangiectasia (˜10⁻²), which was experimentally determined previously (Secomb, T. W. et al., 2003. Proc Natl Acad Sci USA 100, 7231-7234). Once the (Eulerian) velocity field, v_{f}(x) was solved in the model geometry, underlying plasma flow patterns (i.e., the Lagrangian trajectory x(t)) were computed by integrating the differential equation, dx/dt=v_{f}(x) using the fourth-order Runge-Kutta method (Strogatz, S. H., 1994. Nonlinear Dynamics and Chaos with Applications to Physics, Biology, Chemistry and Engineering. Addison-Wesley, Reading). The local fluid particle velocity was estimated using standard 3D interpolation isoparametric scheme inside each 3D finite element (Hughes, T. J. R., 1987. The Finite Element Method-Linear Static and Dynamic Finite Element Analysis. Prentice-Hall, Englewood Cliffs).

Results:

To investigate lymphocyte distribution within the microangiectasias, lymphocyte migration through the inflammatory skin microcirculation was studied. The epicutaneous antigen oxazolone was used in a sheep model to stimulate lymphocyte recruitment out of the skin microcirculation. These focal dilatations were observed at 100-150 μm intervals within the inflammatory microcirculation. Regional efferent lymphocytes were fluorescently labeled and re-injected into the inflammatory microcirculation. These migratory cells were tracked through the skin inflammatory microcirculation using epi-fluorescence intravital videomicroscopy (West, C. A. et al., 2002. Inflamm Res 51, 572-8).

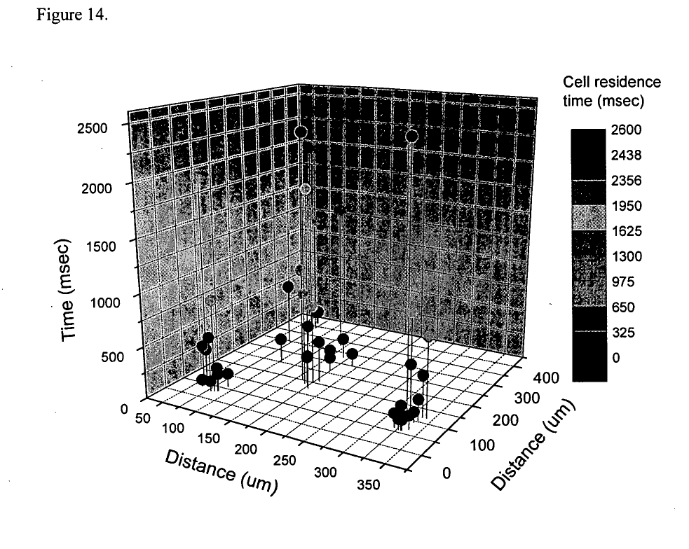

Most lymphocytes slowed to less then 0.3 μm/msec when passing through an individual microangiectasia. A subset of lymphocytes, apparently in the flow stream, slowed to negligible antegrade velocity within the microangiectasia. These lymphocytes paused for a variable length of time prior to returning to their pre-microangiectasia flow velocity. The length of time individual lymphocytes remained at near-zero axial velocity was defined as the cell's residence time. Lymphocyte residence time profiles were distributed over a wide range (FIG. 14). A single labeled lymphocyte population typically demonstrated residence times that varied from 100 msec to more than 10 sec. The frequency of lymphocytes with an increased residence time was estimated to be 2-5%, although the percent varied between individual microangiectasias.

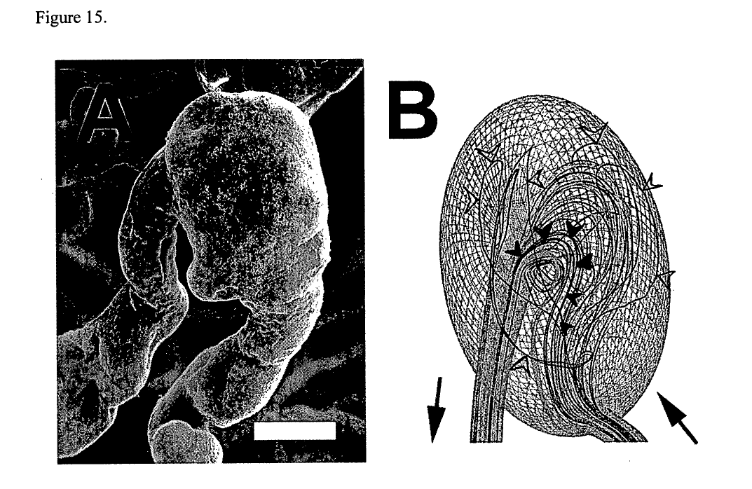

Assuming the lymphocytes remained in the flow stream, simple uniaxial plasma flow could not explain the observed residence times. Further, the constantly oscillating and occasionally retrograding flow paths suggested that tethering of lymphocytes on the endothelium could not explain the lymphocyte residence times. To determine if microhemodynamic conditions within the microangiectasias could account for these observations, blood flow in the microangiectasias was modeled. Studying 3D scanning electron micrographs (FIG. 15A) (Konerding, M. A. & Steinberg, F., 1989. Prog Clin Biol Res 295, 475-80), the basic configuration of the focally dilated microvessel found in the skin was represented as a balloon-like dilatation located at the hairpin turn in the superficial vascular plexus of the skin (Hughes, T. J. R., 1987. The Finite Element Method-Linear Static and Dynamic Finite Element Analysis. Prentice-Hall, Englewood Cliffs; Huebner, K. H., 1975. The Finite Element Method. John Wiley & Sons, New York). Using this geometric model, blood flow patterns were solved numerically (Donea, J. & Huerta, A., 2002. Finite Element Methods for Flow Problems. John Wiley & Sons, New York; Kojic, M. & Bathe, K. J., in press. Inelastic Analysis of Solids and Structures. Springer-Verlag, New York).

A principal finding of the modeling was that flow in the microangiectasia, even when the Reynolds number was less than unity, exhibited remarkably complex patterns due to the abrupt changes in the configuration of the vessel wall (boundary expansions and contractions) (Secomb, T. W. et al., 2003. Proc Natl Acad Sci USA 100, 7231-7234). The core region contained a bundle of streamlines convectively connecting the afferent and efferent microvessels with minimal pathlengths. The region surrounding the core occupied most of the inner volume of the microangiectasias. In this region, the local flow velocity was notably lower than the mean bulk velocity and non-axial (i.e., secondary) flows were present. The intensity of the flow in the outer most region of the microangiectasia, especially near the walls, was substantially lower than in the axially convecting core region. The diminished flow near the walls resulted in significantly decreased wall shear stress (˜1 dyn/cm²) over the entire inner wall surface.

Since the movement of lymphocytes is largely determined by the convective flow patterns of the carrier fluid, cell migration was simulated as a first approximation by tracking the motion of fluid particles (Kojic, M. & Bathe, K. J., in press. Inelastic Analysis of Solids and Structures. Springer-Verlag, New York). The trajectories of the cells, initially distributed uniformly over the cross-sectional area of the afferent vessel, were computed (FIG. 15B). Two types of trajectories were observed: (1) Most of the trajectories were simple and uniaxial (FIG. 15B: filled arrow heads) suggesting that the cells directly passed through the microangiectasia and exited to the efferent vessel; and (2) A few trajectories exhibited polyaxial paths (FIG. 15B: open arrow heads). In particular, the cells that started from the central region of the afferent vessel with relatively high velocity experienced a notable deceleration when they entered the microangiectasia; their paths subsequently diverged from the uniaxial path and deflected to local secondary (e.g., retrograde) flow fields. The trajectories were characterized by swirling and twisting patterns and notable three-dimensionality.

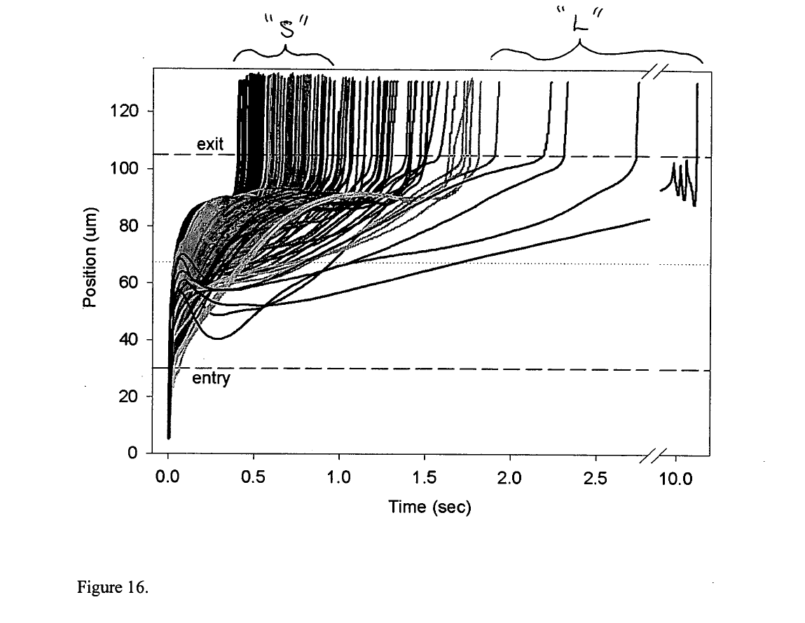

The effect of the swirling/twisting trajectories on lymphocyte residence time within the microangiectasia was quantified by calculating the residence time profiles for individual cells (FIG. 16). The cells demonstrating a simple flow path moved continuously through the microangiectasias. Of note, the axial velocity of the cells with a simple trajectory appeared to be inversely proportional to the increased cross-sectional area of the microangiectasia. This finding was consistent with a rapid, simple, and uniaxial migration path. In contrast, the cells following a complex trajectory demonstrated quite different behavior. These cells initially moved to the midpoint of the microangiectasia, then slowly moved in a retrograde trajectory (negative slope) prior to gradually moving toward the exit of the microangiectasia. Some of these slowly moving cells were trapped by the rotation flow near the exit of microangiectasia manifested by up-and-down (i.e. looping) motion (FIG. 16: labeled “L”). Most important, cells that followed these complex trajectories had significantly prolonged residence times within the microangiectasia. As a consequence, the probability of these cells making contact with the inner walls of the microangiectasia was significantly increased.

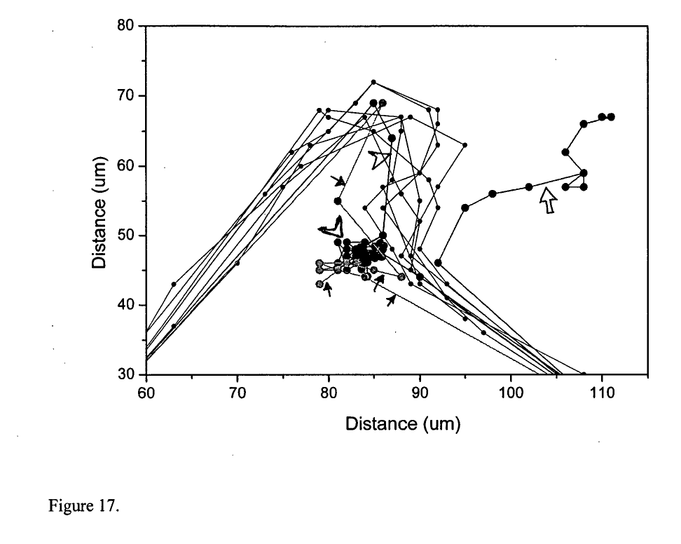

To test the predictions of the flow model, individual lymphocytes were tracked through the individual microangiectasias. The trajectories of lymphocytes within the microangiectasias were consistent with the predictions of the flow modeling. The flow paths of most lymphocytes were simple and uniaxial (FIG. 17: small filled circles). These cells slowed as they passed through the microangiectasia, but demonstrated no loops or retrograde movements. A second population of lymphocytes passing through the microangiectasias demonstrated divergent or polyaxial flow paths. Some of these lymphocytes appeared to move out of the flow stream and pause for varying lengths of time (milliseconds to seconds) before returning to the axial flow stream. The visible oscillation or wobble suggested that the movement of these cells reflected plasma flow and not static adhesion or tethering (FIG. 17: open arrow heads). Other cells demonstrated an increased residence time as a consequence of a looping or apparent retrograde movement (FIG. 17: small arrows). A third subset of lymphocytes diverged from the flow stream, rapidly decelerated, and remained immobilized for the remainder of the tracking period (17: open arrows). The fixed and immobilized appearance of the cells was characteristic of lymphocytes after transmigration (West, C. A. et al., 2001. Am. J. Physiol. Heart Circ. 281, H1742-H1750).

The present intravital microscopy findings in the inflammatory skin microcirculation, demonstrating increased residence times and polyaxial flow paths, are inconsistent with the prevailing assumption that plasma flow is smooth and regular. Based on computational flow analysis, an alternative explanation for these observations is that increased residence times reflect complex pattern of blood flow within microangiectasias. The swirling and looping trajectories of the model predict lymphocyte residence times that are remarkably consistent with those observed in vivo. The computational model's complex trajectories also predict divergent or polyaxial flow paths of migrating lymphocytes. The in vivo confirmation of polyaxial lymphocyte flow paths adds further support for this model of plasma flow patterns within localized segments of the inflammatory microcirculation.

In the context of lymphocyte transmigration, microangiectasia structure appears to have important functional consequences. The acute dilatation of the microangiectasia results in complex plasma flow and polyaxial lymphocyte flow paths. The resulting increase in the residence time and the redistribution of lymphocytes within the vascular segment is likely to increase the probability of functional lymphocyte-endothelial cell interactions. The blood flow pattern within the microangiectasias is also associated with significantly decreased wall shear stress over the entire inner surface. The calculated wall shear stress at the inner wall of the microangiectasias (1-2 dyn/cm²) is compatible with in vitro measures of lymphocyte-endothelial cell adhesivity (Li, X. et al., 2001. In Vitro Cell Dev. Biol. 37, 599-605). These observations suggest that microangiectasia structure, and the complex plasma flow within these microvascular segments, provide a localized, controlled mechanism for the creation of conditions favorable for lymphocyte transmigration.

### Example 4

### Induction of Microangiectasias in the Lung, Gut and Liver

Detailed studies of focal lymphocyte migration were in two organs: lung and gut. These organs were selected to complement the skin because 1) they represent three distinct hemodynamic conditions (Table 1), 2) lymphocyte recruitment in these organs can be readily triggered using peptide hapten antigens (Table 2), and 3) these organs—in addition to the brain—are associated with venular dilatations in the genetic deficiency HHT.

TABLE 1
Study Microcirculations

| Organ | Hemodynamics | Architecture | References |
|---|---|---|---|
| Skin | Systemic | Superficial papillary | Konerding et al. 1992 |
| | cutaneous | loops | |
| Lung | Pulmonary | Peri-bronchiolar | Schraufnagel 1987 |
| | | venules | |
| Gut | Systemic visceral | Submucosal plexus | Konerding et al. 1995 |

In vivo Approach The recruitment of lymphocytes into the tissues of the skin, lung, and gut is triggered using peptide haptens applied to the four organs. In each of the organs, lymphocytic inflammation is characterized by the perivascular infiltration of mononuclear cells. Immunohistochemistry shows that the infiltrate is predominantly comprised of T lymphocytes.

Kinetics of lymphocyte migration: Similar to observations in the skin, quantitative morphometry was used to evaluate lymphocyte recruitment. Variability in technique of TNP administration is assessed with an extended baseline data. Reproducibile tissue conditions was established prior to the pulse-chase experiments.

Topography of lymphocyte migration: The pulse-chase technique is used to follow the migration path of lymphocytes recruited out of the microcirculation. The lymphocytes are identified in vertically sectioned tissue by fluorescence microscopy as focal clusters. Pulse injections is typically performed within the 24 hours prior to the peak of microangiectasia development.

TABLE 2
Model for Microangiectasia

| Organ | Peptide-hapten | Administration/Route* | References |
|---|---|---|---|
| Skin | Oxazalone/ | Epicutaneous application | West et al. 2001(a) |
| | TNP | | |
| Lung | TNP | Intrabronchial instillation | Rawn et al. 2000 |
| Gut | TNP | Intralumenal instillation | Franco et al. 1999 |

The tempo of the lymphocytic inflammation will result in selective lymphocyte accumulation between 72 and 120 hours after stimulation in all organs. The kinetics of lymphocyte recruitment will be reproducible for a given vehicle, but will vary with the use of adjuvants (e.g. ethanol for gut).

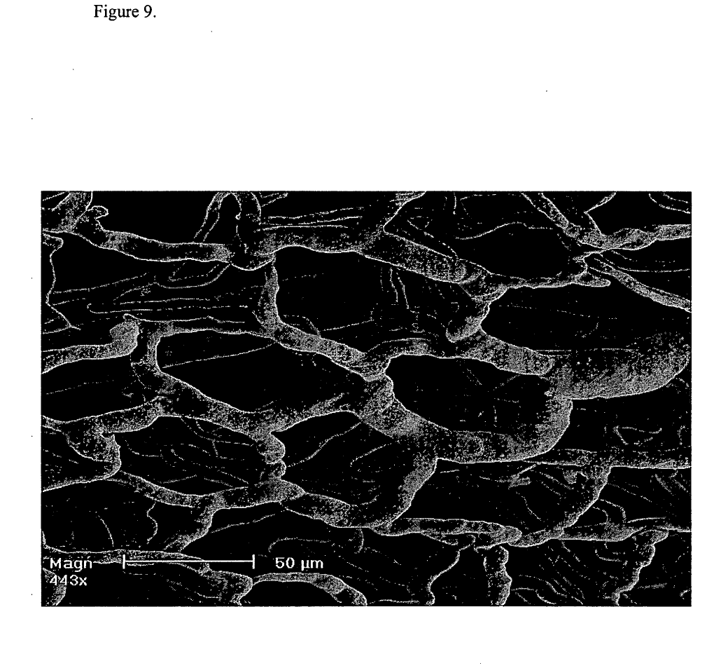

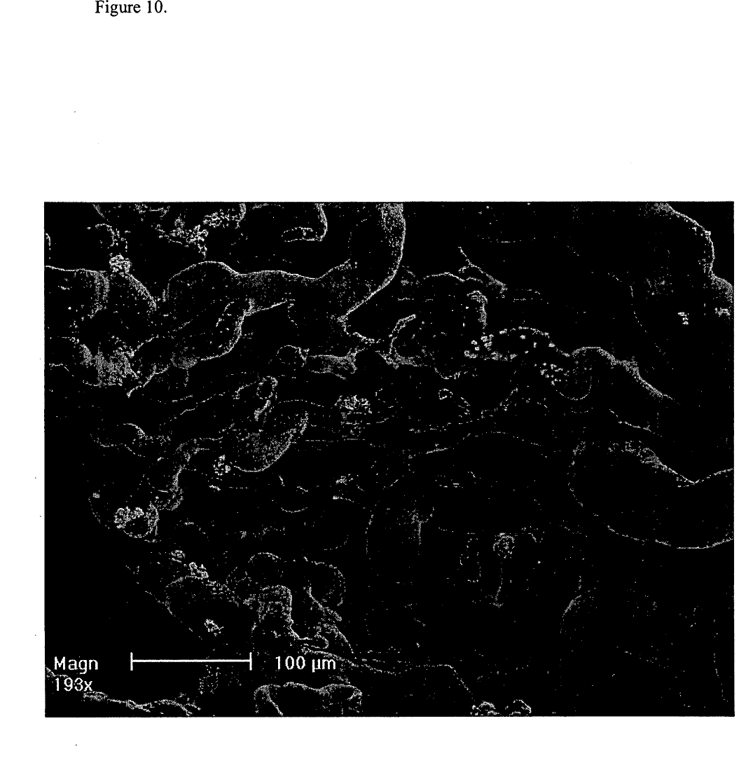

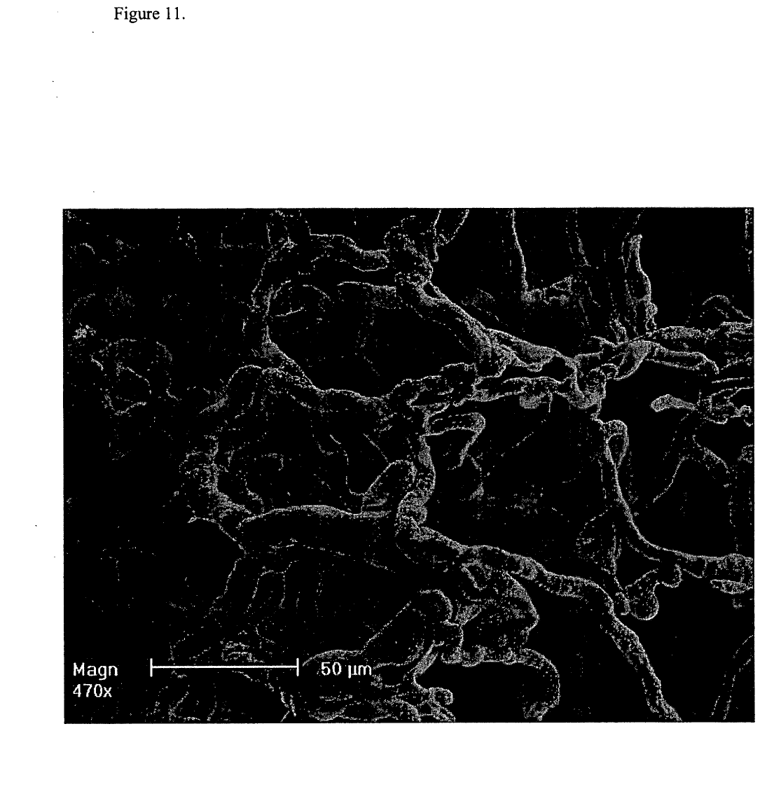

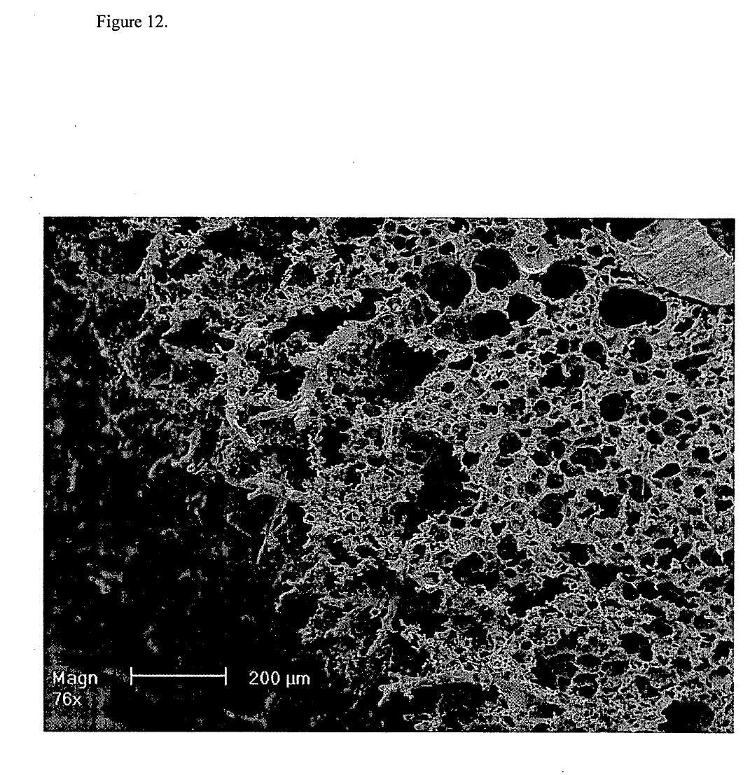

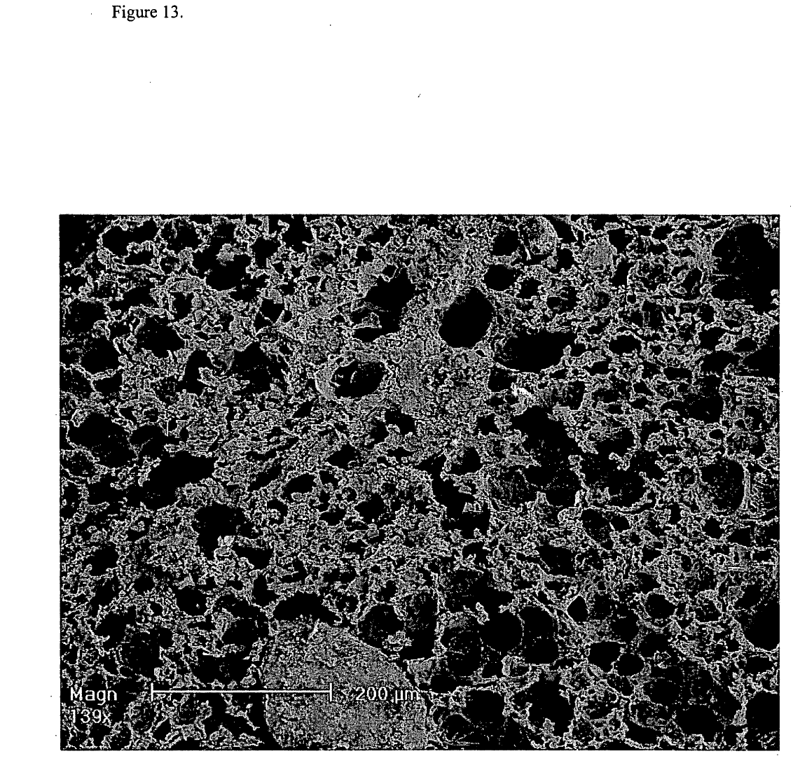

In the lung, fluorescently-labeled lymphocyte clusters will be demonstrated in the central lobules along the peribronchiolar bundles. This region represents the confluence of alveolar capillaries into peribronchiolar venules. The clusters will likely reflect an underlying (potentially stochastic) relationship between alveolar number and microangiectasias. The results of representative analysis in gut and lung are shown in FIGS. 9-13.

### Example 5

### Microvascular Adaptations Associated with Leukocyte Slowing and Transmigration in Hapten-Induced Acute Colitis

In the example disclosed herein, the following abbreviations are used: 3D, 3-dimensional; CFSE, 5-(and-6)-carboxyfluorescein diacetate succinimidyl ester; PBS, phosphate buffered saline; SEM, scanning electron microscopy; TNBS, 2,4,6-Trinitrobenzenesulfonic acid; TNCB, 2,4,6-Trinitrochlorobenzene.

To investigate in more detail the microvascular changes in the inflammatory gut, a murine model of TNBS-induced colitis was investigated. Microvascular dilatations of the mucosal plexus, analogous to microangiectasias, were found to be temporally and spatially associated with TNBS-induced perivascular mononuclear inflammation.

Methods:

Mice. Male Balb/c mice (Jackson Laboratory, Bar Harbor, Me.), 25-33 g, were used in all experiments. The care of the animals was consistent with guidelines of the American Association for Accreditation of Laboratory Animal Care (Bethesda, Md.).

TNBS administration. After the mouse abdomen was sheared and cleansed with water, 36 μl of a 2.5% 2,4,6-Trinitrochlorobenzene (TNCB) (ChemArt, Egling, Germany) in a 4:1 acetone:olive oil solution was sprayed onto a 1.5 cm diameter circular PhastTansfer Filter Paper (Pharmacia, Upsala, Sweden). The TNCB-soaked filter paper was applied to the sheared abdomen and secured with Tegaderm (3M, St. Paul, Minn.) and Durapore Surgical Tape (3M, St. Paul, Minn.). The TNCB patch was removed 24 hours after application. On post-sensitization day six, 125 μl of a 1.75% 2,4,6-Trinitrobenzenesulfonic acid (TNBS) (Sigma, St. Louis, Mo.) in a 50% ethanol solution was instilled into the rectum. Control mice had only the 50% ethanol solution instilled intrarectally.

Clinical assessment of colitis. Total body weight was assessed daily. Activity level and fur ruffling were scored daily on a 0 (normal) to 2 (severe) scale.

Histology. After euthanasia, the tissues were harvested and immediately processed. The tissue was snap-frozen, sliced into 1 cm long segments, coated with tissue freezing medium (Triangle Biomedical Sciences, Durham, N.C.) and placed in 15 mm cryomolds. The cryomolds were frozen in liquid nitrogen-cooled isopentane and stored at −80° C. until sectioning. The slides were stained in Harris hematoxylin (Harris Modified, StatLab, Lewisville, Tex.) for 2 minutes followed by sequential rinses including a brief acid rinse. The slides were counterstained with Eosin Y (Sigma) for 20 seconds then rinsed in ethanol and xylene (Fisher, Fair Lawn, N.J.) followed by mounting with DPX medium (Sigma).

3-Dimensional tissue mounts. Spatial association of the infiltrating cells and the microcirculation was defined by fluorescent vessel painting and topographic mapping. Vessel painting was performed as previously described (Ravnic, D. J. et al., 2005, Microvasc Res In press). After systemic heparinization the aorta was cannulated and perfused with 15 ml of 37° C. phosphage buffered saline (PBS) followed by a buffered 2.5% glutaraldehyde solution (Sigma). The systemic circulation was perfused with 1,1-dioctadecyl-3,3,3,3-tetramethylindocarbocyanine perchlorate (10-25 ml) as described previously. Immediately following tracer infusion, the organs were harvested and the tissues dissected in a PBS bath at 25° C. The prepared specimens were placed between glass slides and fixed in 4% formalin overnight. After a brief rinse with distilled water, the specimens were permanently mounted with Vectashield mounting medium (Vector). The fluorescently labeled microvessels were imaged using a Nikon Eclipse TE2000 inverted epifluorescence microscope using Nikon CFI Plan Fluor ELWD 10×, 20×, and 40× objectives.

Topographic mapping. The tissue mounts of the carbocyanine tracer (excitation 549 nm; emission 565 nm) were counterstained with DAPI (excitation 350 nm; emission 461 nm). Excitation and emission filter wheels with 25 nm band pass filters (Omega, Brattleboro, Vt.) permitted selective visualization of the vessel and infiltrating mononuclear cells to facilitate morphometric thresholding. After Z-axis distance calibration, optical sections were obtained through a 1000 μm×1000 μm digitally superimposed upon the colonic mucosa. The optical sections were imaged and processed with standard Metamorph 6.26 (Molecular Devices, Brandywine, Pa.) filters on a Dell Xeon workstation running Windows XP Professional (Microsoft, Redmond, Wash.). The images were pseudocolored and multi-color images were digitally recombined to confirm the topographic mapping.

Scanning electron microscopy. After systemic heparinization, PBS perfusion and intravascular fixation the systemic circulation was perfused with 10-20 ml of Mercox (SPI, West Chester, Pa.) diluted with 20% methyl methacrylate monomers (Aldrich Chemical, Milwaukee, Wis.) as described previously (Su, M., 2003, J. Cell Physiol. 194, 54-62). After complete polymerization, the tissues were harvested and macerated in 5% potassium hydroxide followed by drying and mounting for scanning electron microscopy. The microvascular corrosion casts were imaged after coating with gold in an argon atmosphere with a Philips ESEM XL30 scanning electron microscope (Eindhoven, Netherlands). Stereo-pair images were obtained using a tilt angle of 6 degrees.

Quantitative morphometry. Diameters were interactively measured orthogonal to the vessel axis after storage of calibrated images, using AnalySIS software (version 2.1). Inter-branch and inter-vessel distances were measured in stereo-pairs using the KS 300 software (Kontron, Eching, Germany) as previously described in detail.

Intravital fluorescence labeling. A 5-(and-6)-carboxyfluorescein diacetate, succinimidyl ester (CFSE) (Molecular Probes, Eugene, Oreg.) labeling solution was prepared in DMSO as described (Becker, H. M et al., 2005, J. Immunol Methods 286, 69-78). The freshly prepared CFSE (400 μl) was injected into the tail vein of an anesthetized mouse over 2-3 minutes.

Intravital microscopy. The exteriorized colon was imaged using a Nikon Eclipse TE2000 inverted epifluorescence microscope using Nikon Fluor 10×, 20×, and 40× objectives. The intravital microscopy was performed by using a custom-machined titanium stage (Miniature Tool and Die, Charlton, Mass.) that directly attached to the objective. The tissue contact area consisted of a 2-mm vacuum galleries that provided tissue apposition to the lens surface without compression of the tissue and with minimal circulatory disturbances. An X-Cite (Exfo; Vanier, Canada) 120 watt metal halide light source and a liquid light guide was used to illuminate the tissue samples. Excitation and emission filters (Chroma, Rockingham, Vt.) in separate LEP motorized filter wheels were controlled by a MAC5000 controller (Ludl, Hawthorne, N.Y.) and MetaMorph software 6.26 (Molecular Devices). The CFSE tracer (ex 480 nm, em 520 μm) was imaged with 25 nm band pass filters (Omega). The 12-bit fluorescent images were digitally recorded (Cool Snap ES, Roper Scientific, Tuscon, Ariz.) with 1392×1040 pixel resolution. Image stacks were routinely created from 5 to 10 minute video sequences. The image stacks were processed with standard MetaMorph filters. Routine distance calibration and thresholding was applied to the “stacked” image sequences.

Time-motion analysis. MetaMorph was used to distance calibrate and time stamp stacked images. Centerline flow was labeled with standard region measurement tools. Time-motion analysis was performed using the kymograph function. Time was assigned to the y-axis and distance to the x-axis on a calibrated kymograph image. Velocity was calculated as a function of Δx/Δy. Time-location analysis was performed using the track points function applied to image stacks. The data was logged into Microsoft Excel 2003 (Redmond, Wash.) by dynamic data exchange.

Results:

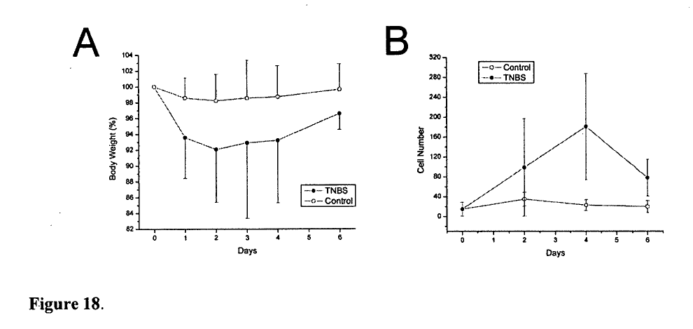

Induction of acute colitis. The transrectal instillation of TNBS produced an inflammatory colitis. Clinical signs of inflammation such as decreased activity (86%) and ruffled fur (63%) were present in the majority of mice. Total body weight, reflecting inflammation-associated obstipation, progressively declined over 96 hours followed by gradual resolution of the weight loss (FIG. 18A). Serial histologic evaluation mirrored the clinical findings. The peak of mononuclear cell infiltration into the colonic wall occurred at 96 hours (FIG. 18B). Based on these findings, most of the subsequent morphologic studies were performed 96 hours after the instillation of TNBS.

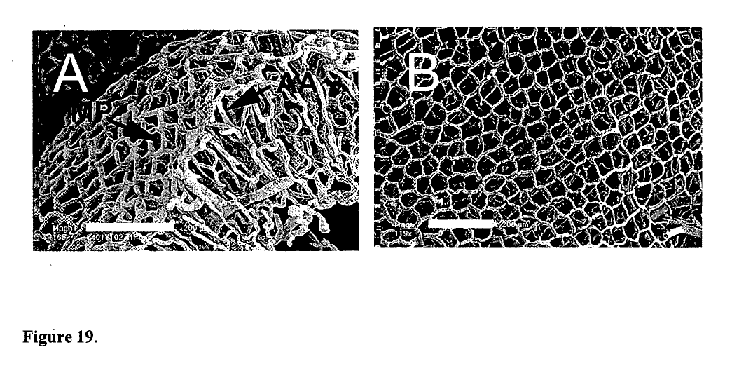

Colonic microcirculation. To define the normal vascular morphology of the mouse colon, corrosion casting and scanning electron microscopy was performed. Similar to findings in humans (Kruschewski, M. et al., 1995, Langenbecks Arch Chir 380, 253-9; Konerding, M. A. et al., 2001, Br J Cancer 84, 1354-62), the lumenal aspect of the colon wall was defined by a polygonal mucosal network surrounding the crypts. A submucosal vascular network near the serosal surface was connected to the mucosal plexus by a dense network of ascending arterioles and parallel descending veins (FIG. 19).

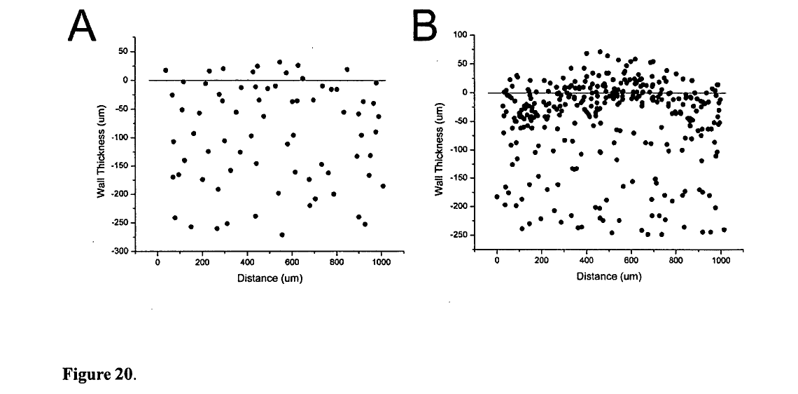

Topography of the cellular inflammation. To determine the spatial relationship between the transmigrating mononuclear cells and the vascular microarchitecture in TNBS-induced colitis, whole mounts of the colon wall were prepared after fluorescent vessel painting and counterstaining with DAPI. 3-D (z axis) optical sections of the colon tissue mounts demonstrated that the mononuclear cells were spatially associated with the mucosal plexus (FIG. 20).

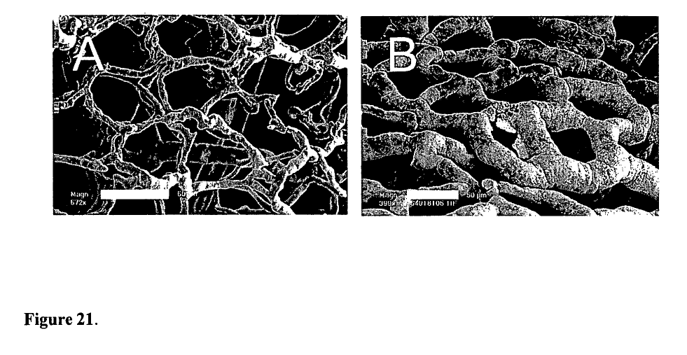

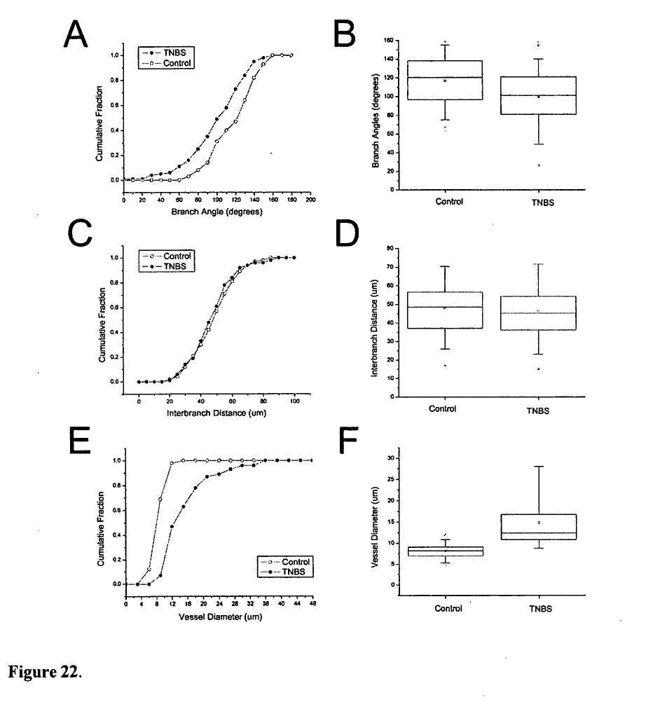

Structural adaptation of the mucosal plexus. The structural adaptation of the mucosal plexus during TNBS-induced colitis was investigated using corrosion casting, 3-D SEM, and quantitative image analysis. Despite topographic variation in the degree of colitis, areas of intense inflammation were associated with marked dilatation of the mucosal capillary plexus (FIG. 21). Quantitative morphology of the mucosal plexus demonstrated no change in branch angle (FIG. 21A, B), or interbranch vessel length (FIG. 22C, D), but a significant increase in vessel diameter (FIG. 22E, F).

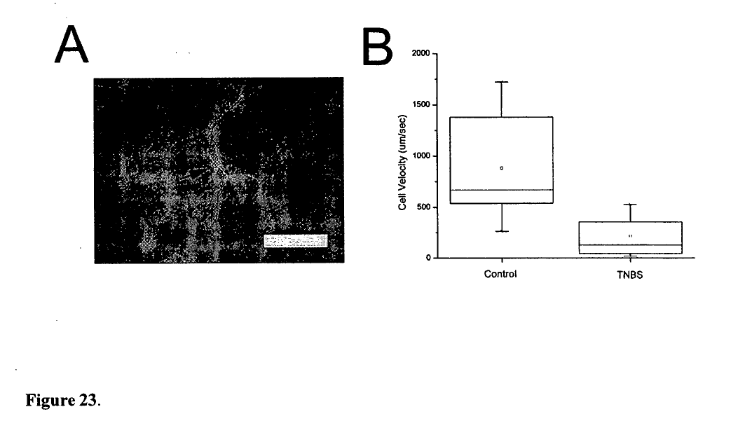

Flow velocity in the mucosal plexus. The functional consequences of the vascular dilatation were investigated by intravital microscopy. The cellular elements of the blood were labeled with intravenous CFSE and examined by epifluorescence intravital videomicroscopy. Videomicroscopy demonstrated notable variability in the instantaneous velocity of individual cells (FIG. 23A). To provide an integrated assessment of cell velocity, the average velocity during mucosal plexus transit was measured. Cell velocity was significantly lower in the inflamed microcirculation (215 μm/sec), than in the normal mucosal plexus (884 μm/sec) (p<0.05) (FIG. 23B).

The present work has defined the normal murine colonic microcirculation: a polygonal mucosal plexus supplied by ascending arterioles and drained by parallel descending veins. Corrosion casting and SEM demonstrated that the induction of TNBS colitis was associated with a significant increase in the diameter of the mucosal plexus. Three lines of evidence suggested that these structural changes were functionally associated with mononuclear cell transmigration. First, the increase in microvessel diameter coincided with the peak of the perivascular mononuclear infiltrate. Second, topographic mapping showed that these structural changes in the mucosal plexus were spatially associated with the mononuclear infiltrate. Third, the dilatation of the mucosal plexus was associated with a significant reduction in monoculear cell velocity during mucosal transit.

The present data suggest that the increased vascularity reflects adaptive structural changes in the mucosal plexus. These changes observed in acute colitis as disclosed herein are likely to be present in chronic disease. Similarly, the dilatation of the mucosal plexus was sufficient to decrease blood flow velocity despite an increase in estimated volumetric flow. Thus, these findings indicate that increased vascularity, increased volumetric flow and decreased flow velocity can coexist in the inflammatory response to TNBS. These microcirculatory findings also suggest that ischemia is unlikely to participate in the early stages of inflammatory colitis.

The present invention shows that lymphocyte adhesion and transmigration occurs in specialized segments exhibiting structural changes that contribute to decreased levels of flow velocity and wall shear stress. More specifically, a decrease in flow velocity in the TNBS-treated mucosal plexus is demonstrated, as well as near-zero flow velocities in many cells passing through the mucosal plexus.

The temporal correlations disclosed herein suggest that the time course of the vascular remodeling may be an important factor in determining the tempo of TNBS-induced colitis; that is, the 3-4 day delay between the instillation of TNBS and the recruitment of the mononuclear cells into the mucosa. The delay may reflect the time necessary for the mural cells to reorganize and/or proliferate sufficiently to produce these changes in microvessel structure, such as endothelial cell mitosis, alterations in mural cell junctions and the enhanced expression of regulatory cell surface molecules.

Finally, the mediators responsible for the vascular changes are likely known endothelial growth factors or inflammatory cytokines. Endothelial growth factors such as the VEGF family have been associated with a variety of chronic inflammatory disorders including inflammatory bowel disease (Shibuya, M., 2001, Cell Struct. Funct. 26, 25-35; Dvorak, H. F. et al., 1995, Int. Arch Allergy Immunol 107, 233-5). In particular, ulcerative colitis and Crohn's disease have demonstrated elevated serum levels of VEGF, basic-FGF and TGF-beta (Kanazawa, S. et al., 2001, Am. J. Gastroenterol 96, 822-8; Griga, T. et al., 1998, Scand J. Gastroenterol 33, 504-8; Griga, T. et al., 1999, Hepatogastroenterology 46, 920-3). Similarly, the apparent interdependence of angiogeneisis and inflammation suggests that cytokines previously associated with inflammation may have a direct influence on vascular remodeling. Cytokines such as G-CSF, GM-CSF, TNF-alpha, IL-1, Il-6 and IL-8 have been associated with a variety of angiogenic effects including endothelial cell proliferation and migration (Ezaki, T. et al., 2001, Am J. Pathol 158, 2043-55); Jackson, J. R. et al., 1997, Inflamm Res. 46 Suppl 2, S129-30). Regardless of the specific mediators, the observation of inflammation-induced structural changes in the colon microcirculation suggests a role for angiogenesis antagonists in the treatment of acute colitis.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for treating a subject having a disease involving inflammation, comprising:
administering an inhibitor of angiogenesis in an amount sufficient to inhibit the formation of acute microvessel dilations.

2. The method of claim 1, wherein the subject has an autoimmune disease.

3. The method of claim 2, wherein the autoimmune disease is an autoimmune disease of the lung.

4. The method of claim 3, wherein the subject has idiopathic pulmonary fibrosis.

5. The method of claim 3, wherein the subject has interstitial lung disease.

6. The method of claim 3, wherein the subject has or is at risk of transplant rejection.

7. The method of claim 2, wherein the autoimmune disease is Crohn's disease.

8. The method of claim 2 wherein the subject has ulcerative colitis.

9. The method of claim 1, further comprising administering an inhibitor of lymphocyte cell-cell adhesion.

10. The method of claim 9 wherein the inhibitor of lymphocyte cell-cell adhesion is selected from the group consisting of an inhibitor of one of LFA-1, CAM 1, and L-selectin.

11. A method for treating a subject having an inflammatory bowel disease, comprising:
administering an inhibitor of angiogenesis in an amount sufficient to treat the inflammatory bowel disease.

12. The method of claim 11, further comprising administering an inhibitor of lymphocyte cell-cell adhesion.

13. The method of claim 12, wherein the inhibitor of lymphocyte cell-cell adhesion is selected from the group consisting of an inhibitor of one of LFA-1, CAM 1, and L-selectin.

14. The method of claim 11, wherein the inflammatory bowel disease is Crohn's disease.

15. The method of claim 11, wherein the inflammatory bowel disease is ulcerative colitis.

16. A method for analyzing microcirculation structural changes, comprising:
labeling systemic microcirculation with a lipophilic carbocyanine tracer and
performing fluorescence microscopy to analyze the microcirculation structural changes.

17. The method of claim 16, where in the structural changes are acute.

18. The method of claim 16, wherein the structural changes are chronic.

19. The method of claim 16, wherein the method is combined with a method of scanning electron microscopy.

20. A method for treating a subject having a disease involving inflammation, comprising:
administering an inhibitor of dilatation in an amount sufficient to inhibit the formation of acute microvessel dilations.

21. The method of claim 20, wherein the inhibitor of dilatation is an inhibitor of angiogenesis.

22. The method of claim 20, wherein the subject has an autoimmune disease.

23. The method of claim 22, wherein the autoimmune disease is an autoimmune disease of the lung.

24. The method of claim 23, wherein the subject has idiopathic pulmonary fibrosis.

25. The method of claim 23, wherein the subject has interstitial lung disease.

26. The method of claim 23, wherein the subject has or is at risk of transplant rejection.

27. The method of claim 22, wherein the autoimmune disease is Crohn's disease.

28. The method of claim 22 wherein the subject has ulcerative colitis.

29. The method of claim 20, further comprising administering an inhibitor of lymphocyte cell-cell adhesion.

30. The method of claim 29 wherein the inhibitor of lymphocyte cell-cell adhesion is selected from the group consisting of an inhibitor of one of LFA-1, CAM 1, and L-selectin.

31. The method of claim 20, wherein the inhibitor of dilatation is an inhibitor of BMPs.
